(19) United States
(12) Patent Application Publication
(10) Pub. No.: US 2007/0238175 A1
(43) Pub. Date: Oct. 11, 2007

(54) Standardization of processes for culturing primary cells

(76) Inventor: Alfred L. Chi, Maynard, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/498,308

(22) Filed: Aug. 1, 2006

Related U.S. Application Data
(60) Provisional application No. 60/744,355, filed on Apr. 6, 2006.

(51) Int. Cl. C12N 5/00; C12N 9/00

(52) U.S. Cl. 435/378; 435/325; 435/183; 435/373

(57) ABSTRACT

The present invention provides a standardized tissue-specific and cell-specific kit and methods for promoting the enrichment and expansion of primary cells in culture while reducing the contamination of unwanted cell types. The present invention further provides the compositions for optimized tissue-specific and cell-type specific dissociation of tissues and inhibition of contaminating cell-types in primary cultures.

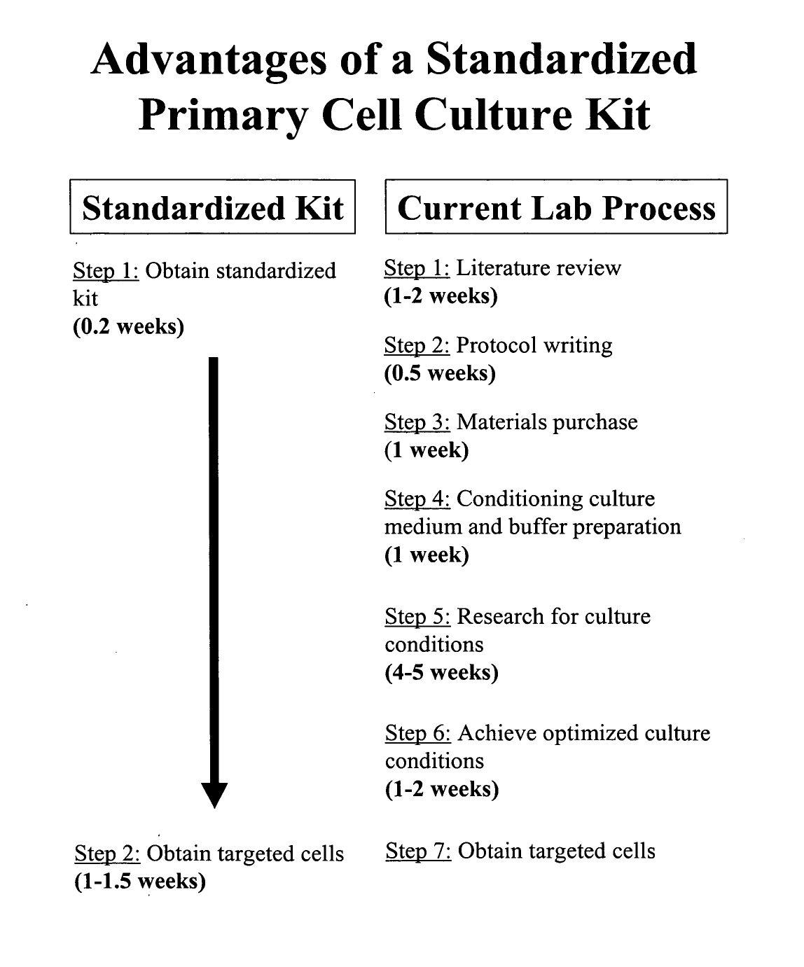

### RELATED APPLICATION

This application claims the benefit of U.S. Provisional Application No. 60/744,355, filed on Apr. 6, 2006. The entire teachings of the above application is incorporated herein by reference.

### BACKGROUND OF THE INVENTION

Primary cell cultures, which are obtained directly from tissues of animals, humans and other species can maintain the differentiated state for a short period (days to weeks) under normal culture conditions. Functionally differentiated primary cell cultures have a limited life span, and, although maintenance of the differentiated properties can be temporarily maintained by culture medium additives, components of the extra-cellular matrix, or by different forms of co-culture, cell specific functions will eventually decline. Cells can proliferate and/or differentiate, both with different limitations, depending on the cell type studied. Because of the meaningful results that can be obtained from primary cells, there is a need for effective and reproducible cell and tissue culture techniques.

There is a need for development of primary cell culture systems to allow for the study of single cellular functions under controlled environmental conditions. In vitro systems share the characteristic that they exclude the influence of other organs and systems, thus providing the possibility of studying direct effects on a cell population. Today's cell culture systems are based on mechanical and/or enzymatic dissociation of the tissue to single cells. Tissue samples are mostly obtained from laboratory animals, biopsy specimens, or samples from surgically removed material, but their use is limited by difficulties in standardization due to variations in sample origin (i.e. genotype, strain/breed, age, etc.), variations in handling, and variations in culture conditions.

Variable culture conditions and the inconsistent handling of organ or tissue samples are two major factors that cause variations in results obtained from primary cell culture experiments. Numerous publications provide protocols for the isolation and culture of different cell types for research; however, conditions and protocols for culturing even the same type of cells from the same type of tissue vary from laboratory to laboratory. Researchers often spend countless hours remedying these issues in laboratories. Data derived from primary cell cultures are often not reliable, not reproducible, and not compatible from experiment to experiment and from laboratory to laboratory. In general, great care should be taken when extrapolating a system from one species to another, from one tissue type to another, and from one cell type to another. A species, tissue, and cell specific, standardized culture system would eliminate these problems.

The development and standardization of species-specific, tissue-specific and cell-specific primary culture systems has become extremely important in biomedical research and drug discovery. Through utilization of primary cultured cells, one can expect to gain new insights while exploring and modulating metabolism and function at the cellular level. Perhaps the most promising features of a standardized primary cell culture system is the possibility of modulating and comparing the metabolic and regulatory pathways of cells of interest and delineating the physiological effects of various compounds and drugs in a standardized way. The refinement and standardization of these experimental tools would be expected to accelerate cellular and genetic research. Furthermore, a reliable cell culture system could be employed as a screening system in various fields of drug discovery, thereby reducing the need for live animals. A species specific, standardized cell culture system, customized for each type of target tissue and cell type, would eliminate the aforementioned problems.

### SUMMARY OF THE INVENTION

The present invention is directed to kits, methods, and compositions for promoting the enrichment and expansion of primary cells in culture. The purpose of the present invention is two-fold; first, it provides for the standardization of tissue preparation and dissociation in order to generate the maximum amount of total viable cells, and second, it provides for the standardization of processes for selectively growing the maximum amount of targeted cells by providing optimal conditions for growth and expansion while minimizing non-target cell growth. Specifically, to obtain a substantially pure cell population, a target cell type is enriched and expanded from a tissue sample by contacting the tissue sample with an enzyme composition to release one or more cell types from the tissue matrix, thereby readying the cells for cell culture; collecting the cell population; and culturing the cells in the presence of a composition that inhibits any contaminating cell growth. The contaminating cells include, but are not limited to, fibroblast cells. The tissue sample is selected from the following tissues: adrenal gland, kidney, bronchia, liver, bone, muscle, brain, ovary, breast, oviduct, cartilage, pancreas, cervix, pituitary gland, colon, prostate, eye, skin, fat, testis, follicles, and thymus. The target cell type can be from each of the aforementioned tissue sample types.

The enzyme composition of the present invention useful for releasing cells from the tissue sample cellular matrix comprises 2 or more components selected from the following: collagenase, collagenase I, collagenase II, collagenase III, collagenase IV, chymotrypsin, elastase, hyaluronidase, trypsin, DNase I, dispase, and papin. This enzyme composition is optimized for use in obtaining a high yield population of viable cells upon collection. The customized enzyme compositions are optimized for tissue samples from the adrenal gland, kidney, bronchia, liver, bone, muscle, brain, ovary, breast, oviduct, cartilage, pancreas, cervix, pituitary gland, colon, prostate, eye, skin, fat, testis, follicles, and thymus.

The composition of the present invention useful for inhibiting contaminating cell growth comprises a combination of at least 2 components selected from the following: trypsin, collagenase, D-valine, cis-OH-proline, hydrocortisone, sodium ethylmercurithiosalicylate, phenobarbitone, fluvastatin, toxin ricin and at least one cell specific antibody. The inhibitory composition can be further made up of a serum substitute and/or buffer(s). The inhibition of contaminating cell growth with the aforementioned composition further promotes target cell enrichment, growth, and expansion since unwanted cell types are inhibited, allowing for establishment and expansion of the desirable primary cell type(s).

In another aspect of the present invention, to produce in vitro primary cell cultures, a primary cell culture system kit is claimed which is made up of: (1) a tissue-specific composition for dissociating cells from a tissue sample; (2) a tissue-specific composition for inhibiting proliferation of contaminating cell types; (3) cell culture media, (4) supplements for cell culture media, such as adenine, cholera toxin, epidermal growth factor, hydrocortisone, antibiotics and streptomycin, insulin, transferrin, highly purified bovine serum albumin, L-ascorbic acid, bovine pituitary extract, basic fibroblast growth factors, sodium selenite, 3,3′5-triiodothyronine, glutamine, dexamethasone, and cytokines; (5) serum and/or plant additives for conditioning the cell culture media, such as fetal-bovine serum, fetal calf serum, animal serum extract, plant additives, formulated serum substitutes (i.e. bovine growth serum (Cat. No. SH30541, HyClone, UT) and bovine serum albumin (BSA); (6) tissue preparation buffers, such as betadine solution, EDTA, EGTA, HEPES, Hanks's salt solution containing fetal bovine serum, phosphate buffered saline (PBS), and basal culture medium containing antibiotics and serum; and, optionally, instructions for using each of the six components of the kit and detailed procedures for culturing desired cells with particular tissues, such as tissue from the adrenal gland, kidney, bronchia, liver, bone, muscle, brain, ovary, breast, oviduct, cartilage, pancreas, cervix, pituitary gland, colon, prostate, eye, skin, fat, testis, follicles, and thymus.

In another aspect of the present invention, a kit for promoting the enrichment and expansion of target cell types from a tissue sample is made up of an enzyme composition for obtaining viable cells from the tissue sample and a composition for enriching the target cell types in culture by inhibiting any unwanted cell types. The kit effectively yields an in vitro cell culture containing an enriched target cell population substantially free of contaminating, or unwanted, cell types. An example of contaminating, or unwanted, cell types is fibroblast cells.

The tissue specific dissociation composition and enzyme composition, used in the kits to dissociate cells from a tissue sample and obtain viable cells, is made up of 2 or more of the following: (1) trypsin, (2) collagenase, (3) D-valine, (4) cis-OH-proline, (5) sodium ethylmercurithiosalicylate, (6) phenobarbitone, (7) fluvastatin, (8) toxin ricin, (9) cell specific antibodies, and (10) hydrocortisone.

The tissue-specific contaminating cell inhibitory composition, used in the kits to inhibit unwanted cell growth and expansion while enriching target cell types, is made up of 2 or more of the following components: (1) collagenase, (2) collagenase I, (3) collagenase II, (4) collagenase III, (5) collagenase IV, (6) chymotrypsin, (7) elastase, (8) hyaluronidase, (9) trypsin, (10) DNase I, (11) dispase, and (12) papin.

The present invention may be achieved and practiced in numerous embodiments, as described herein. In certain embodiments of the invention, the primary cell culture system is customized to provide optimal conditions for enrichment and expansion of a target cell type from a specific tissue. Thus, the present invention provides a tissue-specific and cell-type-specific optimization for the efficient enrichment and expansion of primary cell culture.

### BRIEF DESCRIPTION OF THE DRAWING

FIGURE shows a chart indicating the advantages of using a standardized primary cell culture kit relative to current lab processes.

### DETAILED DESCRIPTION OF THE INVENTION

A description of preferred embodiments of the invention follows.

The following detailed description of the invention is provided to aid those skilled in the art in practicing the current invention. The following descriptions should not be construed to limit the present invention, as modifications and variations may be made in the embodiments discussed by those of ordinary skill in the art without departing from the scope of the present invention.

A number of terms conventionally used in the field of cell culture, cell culture media, and primary cell culture are used. In order to provide a clear and consistent understanding of the specification and claims, including the scope applied to such terms, definitions are provided. It must be noted that as used herein and in the appended claims, the singular forms “a”, “an”, and “the” include plural referents unless the context clearly dictates otherwise. Thus, for example, reference to “a cell” includes a plurality of such cells.

One aspect of the present invention provides for a primary cell culture system kit (also referred to herein as “PrimaCell™”) comprising: (1) a tissue-specific composition for dissociating cells from a tissue sample; (2) a tissue-specific composition for inhibiting proliferation of contaminating cell types; (3) cell culture media; (4) supplements for cell culture media; (5) serum and/or plant additives for conditioning cell culture media; and (6) buffers for tissue preparation. In another aspect, instructions are included for using the six components together as a kit.

The term “cell culture” refers to maintenance and growth, cultivation, or expansion of cells dissociated from the parent tissue in an artificial environment outside of the host's body. This can be termed an in vitro environment. The use of the term “cell culture” is generic and can be used interchangeably with the term “tissue culture”. Both terms, “cell culture” and “tissue culture,” can be used when referring to individual cells, a group of cells, a group or mixture of different or like cell types, tissues, and organs.

The terms “cell culture medium,” “culture medium,” “tissue culture medium,” and the corresponding plural form, “ . . . media,” of each can be used interchangeably and refer to a nutritional solution for cultivating cells, tissues, or organs.

A primary cell culture refers to the cell culture initially derived from the parent tissue prior to any subsequent culture in vitro, or on a cell culture vessel. The cells may be isolated directly from samples of tissue obtained by biopsy, autopsy, surgical or medical procedure, donation, or harvesting. The cells attach and spread across the culture vessel, forming a monolayer of cells. Upon adequate growth and expansion, determined by one skilled in the art, the cells are dissociated from the vessel and diluted into fresh culture vessels. This is known by those skilled in the art as passaging. Subsequent passaging of the primary cell culture yields an expanded culture of cells derived, or originating, from the original tissue.

This standardized primary cell culture system, PrimaCell™, can be developed and customized for any species and any type of cell from all tissues. A representative list of species for which PrimaCell™ is applicable are: amphibian, avian, human, mouse, rat, and plant. A representative list of organs and tissues for which PrimaCell™ is applicable are: adrenal glands, airway tissues, bone, brain, breast, cartilage, cervix, colon, eye, fat, follicles, kidney, liver, muscle, ovary, oviduct, pancreas, pituitary gland, prostate, skin, testis, and thymus. A representative list of primary cells for which PrimaCell™ is applicable are: adipose cells, adrenocortical cells, bursal cells, epithelium, endothelium, granule cells, hepatocytes, keratinocytes, leukocytes, melanocytes, muscle cells, osteoblasts, oviduct cells, pituitary cells, rigmented retinal cells, spleen cells, tendon cells, and thymic cells. Another aspect of the invention would include instructions on how to use the primary cell culture system with each of the aforementioned tissue and cell types.

The tissue-specific composition for inhibiting proliferation of contaminating cell types (also referred to herein as “FibrOut™”) consists of several biochemical compounds and reagents which prevent fibroblast, or non-target cell, overgrowth and increase target cell growth during primary cell culture. The composition comprises 2 or more components selected from the group consisting of: trypsin, collagenase, D-valine, cis-OH-proline, sodium ethylmercurithiosalicylate, phenobarbitone, fluvastatin, toxin ricin, hydrocortisone, and cell specific antibodies. Trypsin is used at a concentration ranging from 0.1% to 15%. Collagenase is used at a concentration ranging from 0.01% to 10%. D-valine is used at a concentration ranging from 1.0 nM to 50 μM. Cis-OH-proline is used at a concentration ranging from 1.0 nM to 3.0 mM. Sodium ethylmercurithiosalicylate is used at a concentration ranging from 10.0 nM to 1.0 mM. Phenobarbitone is used at a concentration ranging from 1.0 nM to 10.0 μM. Fluvastatin is used at a concentration ranging from 1.0 nM to 1.0 μM. Toxin ricin is used at a concentration ranging from 1.0 nM to 10.0 μM. Hydrocortisone is used at a concentration ranging from 0.1% to 5%. The antibodies used are selected from penicillin, streptomycin, fungizone, and gentamycin. FibrOut™ is customized to be tissue- and cell-specific.

The tissue-specific composition for dissociating cells from a tissue sample (also referred to herein as “OptiTDS™”) comprises 2 or more components selected from the group consisting of: collagenase, collagenase I, collagenase II, collagenase III, collagenase IV, chymotrypsin, elastase, hyaluronidase, trypsin, DNase I, dispase, and papin. Collagenase is used at a concentration ranging from 0.01% to 10%. Collagenase I is used at a concentration ranging from 0.01% to 15%. Collagenase II is used at a concentration ranging from 0.01% to 15%. Collagenase III is used at a concentration ranging from 0.01% to 15%. Collagenase IV is used at a concentration ranging from 0.01% to 15%. Chymotrypsin is used at a concentration ranging from 0.03% to 10%. Elastase is used at a concentration ranging from 0.01% to 9%. Hyaluronidase is used at a concentration ranging from 0.01% to 15%. Trypsin is used at a concentration ranging from 0.5% to 15%. DNase I is used at a concentration ranging from 0.01% to 5%. Dispase is used at a concentration ranging from 0.01% to 5%. Papin is used at a concentration ranging from 0.05% to 10%. OptiTDS™ is customized to be tissue- and cell-specific. The enzymes making up the composition can be generated as recombinant enzymes or obtained from bovine pancreas, Bacillus polymyxa, or Clostridium Histolyticum.

The cell culture media is a basal culture and growth media for cell culture. It is made up of a special formulation for optimized tissue- and cell-specific target cell growth. The basic cell culture media comprises one or more components selected from DMEM, RPMI 1640, F-10, F-12, McCoy's, NCTC series, MEM, Waymouth's and William's, Medium 199 and TC-100. The culture media of the present invention are typically sterilized to prevent unwanted contamination of microorganisms.

The cell culture media supplements are made up of growth factors, cytokines, and other growth supplements that condition the cell culture media for optimized cell- and tissue-specific target cell growth. The supplements used comprise several components, such as adenine, cholera toxin, epidermal growth factors, hydrocortisone, antibiotics and streptomycin, insulin, hydrocortisone, transferrin, highly purified bovine serum albumin, L-ascorbic acid, bovine pituitary extract, basic fibroblast growth factors, sodium selenite, 3,3′5-triiodothyronine, glutamine, dexamethasone, and/or cytokines.

The serum and/or plant additives are batches of animal serum and/or plant additives customized for the tissue- and cell-specific growth of target primary cells. This comprises one or more of the selected components from fetal bovine serum, fetal calf serum, animal serum extract, plant additives, formulated serum substitutes (i.e. bovine growth serum, Cat No. SH30541, HyClone, UT) and bovine serum albumin.

The buffers for tissue preparation are various sterilized and purified buffers and solutions used during tissue dissociation. The buffers are made up of one or more of the following: NaCl, KCl, CaCl₂, MgCl₂, MgSO₄, Na₂HPO₄, KH₂PO₂, NaHCO₃, NaH₂PO₄, glucose, and phenol red. They are formulated to be tissue- and cell-specific. The tissue preparation buffers can include betadine solution, Hank's salt solution, containing fetal bovine serum, PBS, and basal culture medium, containing antibiotics and serum.

The combined use of OptiTDS™ and FibrOut™ promotes the enrichment and expansion, or increase in number, of target cell types from a tissue sample, while inhibiting contaminating, or untargeted, cell types. The term “enrich” and “enrichment” refers to the state by which a target cell type is the predominate, or majority, cell type within a population of cells. The term “contaminating” or “untargeted” or “unwanted” cell refers to any cell type whose growth and expansion is not desirable in the primary cell culture and can be used synonymously.

As described herein, using the kits and components of the present invention, a high yield of viable cells is obtained from the tissue sample, and addition of fibroblast, or other contaminating cell, inhibitors allows for the enrichment and expansion of the target cell type(s). As described herein, such methods provide for a substantially pure cell population within the primary cell culture. The term “substantially pure,” as used herein, refers to the predominant presence of target cell types within the primary cell culture with a low enough concentration of unwanted, or untargeted, cell types such that they will not interfere with any subsequent procedures or analyses performed on the primary cell culture population.

Tissues should be handled in standard sterile techniques, as practiced by those skilled in the art. The tissue should be cut into small pieces, when applicable, using sterile surgical instruments and washed before the dissociation procedure. Alternatively, for blood vessels, they should be flushed with a syringe. During the dissociation procedure, the tissue(s) is/are incubated in the tissue-specific composition for dissociating cells (e.g., OptiTDS™) for about 2 to 24 hours at about 25° C. to 37° C., followed by washes, centrifugation, and/or straining to obtain a mixture of cells substantially free of larger cell clumps and/or tissues. The term, “dissociation,” refers to the breaking apart, or dissolution, of the extracellular matrix holding adjacent cells together within a tissue, such that individual cells are obtained with no, or very few, adjacent cells still attached.

The isolated primary cells can be cultured at desired densities. Mammalian cells are typically cultured in a humidified incubator at 37° C. with about 3-10% carbon dioxide in the air. The primary cells are cultured in complete media (basal culture media, serum, media supplements, and fibroblast (or other contaminating cell) growth inhibitors (FibrOut™). Upon desired confluency, the cells are passaged (subcultured) and/or cryopreserved by standard techniques in the art.

In certain aspects of the present invention, a layer of feeder cells may be desirable to one skilled in the art. The feeder cells can be obtained by irradiating 3T3 cells at about 30 Gy, or irradiating human fibroblasts at about 70 Gy, or chemical treatment of 3T3 cells or human fibroblasts (i.e. Mitomycin C treatment), prior to seeding the primary cells on top of the monolayer of feeder cells.

In another aspect of the present invention, tissue culture plates (or Petri dishes) are coated with gelatin prior to use. The bottom of the culture dish is covered with about a 1.5% gelatin solution in phosphate buffered saline (PBSA) and then incubated. Following incubation for a period of time sufficient for adequate coating of the dish with gelatin, the solution is removed, or aspirated, from the dish, and culture media and cells can be added and cultured.

The following examples are illustrative only, and not limiting of the remainder of the disclosure in any way whatsoever.

### EXAMPLES

### Example 1

### Growth of Mouse Vascular Endothelial Cells

The following protocol is developed for the attachment and growth of normal adult mouse vascular endothelial cells using the primary cell culture system of the present invention.

### Mouse Endothelium PrimaCell™: Vascular Endothelial Cells

### I. General Description:

This protocol is developed for attachment and growth of normal mouse vascular endothelial cells from adult mouse endothelium tissues with the Mouse Endothelium PrimaCell™ system. This system provides an optimal condition of tissue dissociation, using the Endothelium OptiTDS™, that routinely yields 5-7 times more cells than most of the tissue dissociation protocols published in the literature (Cells are visualized and counted with a hemocytometer under light microscopy). In addition, this system ensures a high viability of the target cells with improved gradient contained in the provided culture medium. With the described fibroblast inhibitory system described herein (e.g., FibrOut™) cells are grown with a minimized amount of contamination of fibroblastic cells.

Endothelial cells make up a single cell layer at the inner surface of all blood vessels. Historically, the vessels most commonly used to obtain cultured endothelial cells are the bovine and mouse aorta, bovine adrenal capillaries, rat and mouse brain capillaries, human umbilical veins, and human dermal and adipose capillaries. Although all endothelia share some properties, significant differences exist between the endothelial cells of large and small blood vessels.

Preparation of the aorta, capillaries or blood vessels for cell culture is usually started within 1-2 h of removal. If this is impossible, fine cutting of the vessel into 10-15 mm pieces with scalpels and storage overnight at 4° C. in washing medium (see below) can also prove successful.

The Mouse Endothelium PrimaCell™ system applies to most types of endothelium tissues from mice at E16 or above, although mice at E20-22 or 2-3 weeks are recommended for convenience of procedures and yielding maximum amount of viable target cells. Endothelium samples containing pathological organisms (virus, parasites, etc.) or tumors may not be suitable for this system.

### 1.1 Components of Mouse Endothelium PrimaCell™ System

Endothelium Tissue Dissociation System, Endothelium OptiTDS™, (2 aliquots)—A mixture of 0.5% collagenase I, 0.1% collagenase II, 0.3% collagenase IV, 0.5% dispase.
Endothelium OptiTDS™ Reconstitution Buffer, (2×1 ml). 0.01% HCl; 5% BSA
Endothelium OptiTDS™ Digestion Buffer, (2×9 ml). 10.0 mM NaCl; 0.5 mM KCl; 0.15 mM CaCl₂; 0.2 mM MgCl₂; 0.10 mM MgSO₄; 2.0 mM Na₂HPO₄; 0.05 mM KH₂PO₂; 0.4 mM NaHCO₃; 1.0 mM glucose.
Endothelium Tissue Washing Medium, (1×100 ml)—Basal Endothelium PrimaCell™ Culture Medium with 5% FBS, 200 u/ml of penicillin, 200 μg/ml of streptomycin, and 50 μg/ml of gentamycin.
Mouse Endothelium Fibroblast Growth Inhibitors, Endothelium FibrOut™ (5×200 μl)—A mixture of 5 nM cis-OH-proline, 0.3% collagenase, 1 nM D-valine, and formulated serum substitutes (bovine growth serum, Cat No. SH30541, HyClone, UT).
Mouse Endothelium PrimaCell™ Basal Culture Medium, (5×100 ml)—Modified formulation based on medium 199 and DMEM medium (mix of equal volume of both)
Mouse Endothelium PrimaCell™ Medium Supplements, (5×1.0 ml)—A mixture of 10 ng/ml EGF, 10 ng/ml VEGF, 1 ng/ml Heparin, and 2 μg/ml mouse serum extracts, which were lyophilized powder from 24 hour-10% charcoal-dextran stripped adult mouse serum.
Mouse Endothelium PrimaCell™ Serum, (1×50 ml)—Highly purified 24 hour-10% charcoal-dextran stripped fetal calf serum.

### 1.2 Required Materials but not Provided

1. 70% sterile ethanol

2. 1.5% gelatin solution in PBSA

3. PBSA (PBS containing 0.5% bovine serum albumin)

4. Culture dishes

5. Pasteur pipettes and 10-ml pipettes

6. Test tubes, 12 and 50 ml

7. Two clamps or hemostats, 25 mm

8. Sharp scissors, 50 mm

### II. Procedures

### 2.1 Material Preparation

Materials used in this experiment should be sterile or autoclaved to prevent contamination. To enhance cell attachment to the culture dishes, fresh gelatin-coated plates or culture dishes are recommended (see below for treatment of culture dishes).

### 2.2 Treatment of Culture Dishes

1. To tissue-culture-grade Petri dishes, 10 cm, or 75-cm² flasks, add 10 ml or appropriate volume adequate to cover the bottom area, of 1.5% gelatin in PBSA.
2. Incubate the plates or dishes overnight in the gelatin solution at room temperature in the cell culture hood.
3. Remove the gelatin solution, without washing, and add the complete endothelium culture medium containing serum and supplements, and incubate the mixture until the cells are ready for culture. Pre-coated gelatin plates or dishes must be used within 72 hours from aspirating gelatin solution.

### 2.3 Endothelium Tissue Preparation

1. Mice at ages of E20-22 or 2-3 weeks are recommended for convenience of procedures and yielding the maximum amount of viable target cells. Mice are sacrificed by CO₂ narcosis.
2. Aseptically isolate blood vessels, preferably in 10-15 mm sections, approximately 5 mm in diameter. If asepsis cannot be guaranteed, clamp both ends of the blood vessel.
3. Incubate blood vessels for up to 10 min in Endothelium Tissue Washing Medium to prevent infection. (This procedure will not affect endothelial cell viability.)
4. Incubate tissues in 10 ml 70% sterile ethanol for 30 sec.
5. Rinse tissue twice in Endothelium Tissue Washing Medium for 5 min each and keep on ice.

### 2.4 Tissue Dissociation

### 2.4.1 Mouse Endothelium OptiTDS™

In primary cell culture, there are several important factors that can affect the yield and viability of cells. These include the type of tissues, origin of species, age of the animal used, enzymes, culture mediums and growth supplements. The Mouse Endothelium Tissue Dissociation System, OptiTDS™, is suited for optimal dissociation of normal adult endothelium tissues to yield the maximum number of single endothelial cells.

### 2.4.2 Enzyme Compositions

Dispase: from Bacillus polymyxa

Collagenase I: from Clostridium Histolyticum

Collagenase II: from Clostridium Histolyticum

Collagenase IV: from Clostridium Histolyticum

### 2.4.3 System Components

Endothelium Tissue Dissociation System, OptiTDS™, 2 vials.

Endothelium OptiTDS™ Reconstitution Buffer, (2×1 ml).

Endothelium OptiTDS™ Digestion Buffer, (2×9 ml).

### 2.4.4 Procedures for Tissue Preparation and Dissociation

1. Prepare fresh enzyme working solutions. To each vial of Endothelium Tissue Dissociation System, OptiTDS™, add 1.0 ml of the Endothelium OptiTDS™ Reconstitution Buffer. Mix well.
2. Add 1.0 ml of the fresh enzyme working solution to one vial of Endothelium OptiTDS™ Digestion Buffer (9.0 ml). Warm the diluted Endothelium OptiTDS™ working solution at 37° C. for 10 min prior to use. For optimal results, we recommend using 2-3 gram tissue samples per 10 ml diluted Endothelium OptiTDS™ working solutions.
3. Ligate one end of a 10-cm section of blood vessel 2-10 mm in diameter to a 5-ml plastic syringe.
4. Run the fresh enzyme working solution through the blood vessel until it appears at the bottom end, clamp that end with a hemostat, and incubate the vessel containing the fresh enzyme working solution at room temperature for 30 min, with rocking.
5. Cut the vessel above the clamp with sharp scissors, and collect the fresh enzyme working solution in a 10-cm Petri dish.
6. Rinse the lumen of the vessel with 10 ml of Endothelium Tissue Washing Medium, and add this to the enzyme working solution collected from step 5.
7. Repeat procedure 3-5 until all blood vessels are processed and collect all enzyme working solution with cells.
8. Collect cells by centrifugation at 350 g, washing cells with 10 ml PBS or complete culture medium twice. At the end of washing process, collect cells and dilute cells in 0.5-1.0 ml complete culture medium.
9. Count viable cells.
10. Seed cells at 37° C. in Complete Mouse Endothelium PrimaCell™ I Culture Medium at desired densities.

### 2.4.5 Storage:

Tissue dissociation systems should be reconstituted before use and can only be stored for 2-4 days at 4° C. For long-term use, it should be aliquotted and stored at −20° C. Avoid repeated freeze-thaw cycles.

### 2.5 Culture of Vascular Endothelial Cells

### 2.5.1 Medium Preparation.

Thaw out the Mouse Endothelium PrimaCell™ Medium Supplements and Mouse Endothelium PrimaCell™ Serum on ice. To every 100 ml Mouse Endothelium PrimaCell™ Basal Culture Medium, add one vial of Mouse Endothelium PrimaCell™ Medium Supplements; 10 ml Mouse Endothelium PrimaCell™ Serum, and one vial of Mouse Endothelium Fibroblast Growth Inhibitors, Endothelium FibrOut™. Mix thoroughly and warm the complete medium at a 37° C. water bath for 10 min prior to use.

### 2.5.2 Primary Culture Protocols.

1. Resuspend the final pellet collected from the last step described in 2.3.4 in complete Mouse Endothelium Culture Medium, and seed the cells into freshly prepared gelatin-coated dishes or flasks. Approximately use cells derived from one 10-15 cm section of blood vessel, 5 mm in diameter, per 72-cm flask or 10-cm-diameter dish.
2. Subculture by conventional trypsinization methods.

### 2.5.3 Subculture and Propagation

1. Gently rinse the culture dish twice with 1×PBS.
2. Add 3 ml of 0.25% trypsin/0.1% (2.5 mM) EDTA and incubate at 37° C. Examine the dish under phase microscopy every 5 min to detect cell detachment.
3. When most cells have detached, add 10 ml complete endothelium culture medium to terminate the trypsin activity.
4. Pipette the contents of the dish to ensure complete endothelial cell detachment.
5. Centrifuge the supernatant with the cells for 5 min at 350 g.
6. Aspirate the supernatant, resuspend the cells in a complete growth medium, and re-plate at 2-4×10⁵ cells per 100-mm dish.
7. Refeed the culture twice a week with complete endothelium culture medium.

### III Cryopreservation

Cryopreservation is often necessary to maintain large quantities of cells derived from the same tissue sample; the best results are reported when cells from pre-confluent primary cultures are used.
1. Detach cells, as for subculturing, and centrifuge at 350 g for 10 min.
2. Resuspend cells in complete culture medium with serum and count.
3. Dispense aliquots of 2×10⁶ cells/ml in complete growth medium with additional 15% FCS and 10% glycerol into cryopreservation tubes.
4. Equilibrate at 4° C. for 1-2 h.
5. Freeze cells with a freezing apparatus at a cooling rate of 1° C. per min.
6. To recover cells:
   (i) Thaw cryotubes quickly in a 37° C. water bath.
   (ii) Dilute cells tenfold with medium.
   (iii) Centrifuge cells, and resuspend them at an appropriate concentration in the desired culture medium. Seed culture vessel.

### Mouse cells can be grown in all media for 4-7 weeks and can be subcultured only 4-5 times.

### IV Fibroblast Contamination

The Mouse Endothelium PrimaCell™ system includes a fibroblast elimination system, the Mouse Endothelium Fibroblast Growth Inhibitors, Endothelium FibrOut™. It contains a mixture of cis-OH-proline, collagenase, D-valine, and formulated serum substitutes. This system can effectively eliminate Endothelium fibroblast contamination while having no affect on the behavior of endothelial cells.

### V Confirmation of Vascular Endothelial Cells

Vascular endothelial cells are typically identified by: the production of factor VIII, angiotensin-conversion, the uptake of acetylated low-density lipoprotein, the presence of Weibel-Palade bodies, and the expression of endothelial-specific cell surface antigens.

### VI References:

1. Zetter B R, “The endothelial cells of large and small blood vessels”, Diabetes, 30(Suppl 2):24-8 (1981).
2. Kern P A, Knedler A, Eckel R H, “Isolation and culture of microvascular endothelium from human adipose tissue”, J Clin Invest, 71(6):1822-9 (1983).
3. Davison P M, Bensch K, Karasek M A, “Isolation and long-term serial cultivation of endothelial cells from the microvessels of the adult human dermis”, In Vitro, 19(12):937-45 (1983).
4. Jaffe E A, Nachman R L, Becker C G, Minick C R, “Culture of human endothelial cells derived from umbilical veins. Identification by morphologic and immunologic criteria”, J Clin Invest, 52(11):2745-56 (1973).
5. Folkman J, Haudenschild C C, Zetter B R, “Long-term culture of capillary endothelial cells”, Proc Natl Acad Sci USA, 76(10):5217-21 (1979).
6. Bowman P D, Betz A L, Ar D, Wolinsky J S, Penney J B, Shivers R R, Goldstein G W, “Primary culture of capillary endothelium from rat brain”, In Vitro, 17(4):353-62 (1981).
7. Booyse F M, Sedlak B J, Rafelson M E Jr, “Culture of arterial endothelial cells: characterization and growth of bovine aortic cells”, Thromb Diath Haemorrh, 34(3):825-39 (1975).
8. Kobayashi M, Inoue K, Warabi E, Minami T, Kodama T, “A simple method of isolating mouse aortic endothelial cells”, J Atheroscler Thromb, 12(3): 138-42 (2005).

### Example 2

### Growth of Mouse Epidermal Keratinocytes

The following protocol is developed for the attachment and growth of normal mouse epidermal keratinocytes using the primary cell culture system of the present invention.

### Mouse Skin PrimaCell™ II: Epidermal Keratinocytes

### General Description:

Keratinocytes have been widely used as target cells for testing the activity of oncogenes in epithelial neoplasia. Many experimental studies have utilized cultured mouse skin Keratinocytes, where in vitro results can be analyzed in the context of a substantial experience in carcinogen-induced mouse skin tumors. More recent experiments have employed Keratinocytes derived from human skin, oral cavity, or cervix, where results can be directly extrapolated to cancers or warts originating in the corresponding epithelia. Several laboratories have utilized mouse or rat Keratinocytes in analyses of oncogenes.

The Skin PrimaCell™ II system is suited for culturing epidermal Keratinocytes from the skin of mice.

The Mouse Skin PrimaCell™ II system includes:
Mouse Skin Tissue Dissociation System (2×1 ml) (2% Trypsin from Bovine Pancreas; 1% Dispase from Bacillus polymyxa; 0.5% Collagenase I from Clostridium Histolyticum; 0.1% Collagenase II from Clostridium Histolyticum; 0.2% Collagenase IV from Clostridium Histolyticum)
Specific Skin Fibroblast Growth Inhibitors—FibrOut™ (1.0 ml) (5 nM EGTA, 2 nM EDTA, 0.5 μg/mL hydrocortisone, 1.5 nM D-vlaine).
Medium A Supplements (5×10 ml): 18 mM adenine, 0.1 nM cholera toxin, 7 ng/ml EGF, 0.5 μg/ml hydrocortisone, 10% FCS, and antibiotics ((penicillin, 100 U/ml), and streptomycin (50 μg/ml)).
Medium B Supplements (5×10 ml): 5 μg/ml insulin, 0.5 μg/ml hydrocortisone, 0.1 ng/ml EGF, 20 μg/ml transferrin, highly purified bovine serum albumin, and 50 μg/ml L-ascorbic acid, 1.3 mM CaCl₂, 0.1% Bovine Pituitary Extract, antibiotics (penicillin (100 U/ml) and streptomycin (50 μg/ml)).
10% Betadine Solution (20 ml)
10% Trypsin (200 μl)

### Required Materials (but not Provided):

1. Medium A: Ham's F12:DMEM (high calcium) 1:3 mixture.

2. Medium B: Plain MCDB153 (calcium free).

3. Centrifuge tube, 50 ml

4. Nylon gauze

5. Cell Strainer (BD Bioscience)

6. Petri dishes, bacteriological grade, 100 mm

7. Scalpels, curved forceps

8. 3T3 cells or human fibroblasts (optional)

9. 70% ethanol, sterile

10. 0.05% EDTA (pH 7.4), sterile

11. CaCl₂, sterile.

12. PBSA (PBS containing 10% BSA), sterile.

### Principle

Separate the epidermis from the dermis enzymatically, disaggregate the Keratinocytes, and seed them in a serum-free medium or on a growth-arrested feeder layer. Separation of the epithelial compartment from the underlying connective tissue is done by enzymatic digestion using tissue-specific dissociation systems which are mixtures of several tissue dissociation enzymes. The Skin Dissociation System is developed for optimal separation of Keratinocytes, which contains trypsin, dispase type II, and Collagenases. The isolated epithelium is further dispersed by additional incubation in medium containing Skin Dissociation System, or mechanically, by pipetting, after which it is filtered through nylon gauze and propagated in a serum-free, low-calcium medium or on growth-arrested feeder cells by using different media formulations. Subpopulations of Keratinocytes with stem cell characteristics can be isolated due to their selective attachment to basement membrane constituents.

### Protocol

### Specimens:

1. Mouse Skin: Obtain foreskin (neonatal as well as juvenile), the most frequent laboratory source for mouse skin, or trunk skin obtained from surgery or post mortem (up to 48 h). Keratinocytes derived from foreskin seem to attach and proliferate better than cells obtained from adult skin.
   Mouse Skin: Newborn (prefer 1-2 days) mouse epidermis yields a large number of cells (5-10×10⁶/epidermis), with a 30-40% plating efficiency. Mice are sacrificed by CO₂ narcosis 1-4 days postpartum (prior to the appearance of hair). Using an aseptic technique, limbs and tails are amputated, a longitudinal incision is made from tail to snout, and skin is peeled off the carcass using forceps.
2. Incubate skin biopsies for up to 10 min in 10% betadine solution to prevent infection. (This procedure should not affect Keratinocyte cell viability.)
3. Rinse tissue twice in PBSA for 10 min each and keep on ice.

### Epidermal Separation:

4. Rinse surgical instruments in sterile PBS. Cut off tail and paws at the “ankles” and cut a line through the pelt from tail to head.
5. Gently peal off pelt from the body, rinse in PBS, and place in 100 mm dish with 10 ml Skin Dissociation System solution (1:10 dilution in PBS from the stock).
6. Incubate tissue with Skin Dissociation System by one of the following steps:
   (a) Rapid dissociation: Float skin samples in Skin Dissociation System solution for 2-3 h at 37° C. This works particularly well also with skin at full-thickness.
   (b) Slow dissociation: Float the samples on ice-cold Skin Dissociation System at 4° C. for 15-24 h.
7. Monitor the separation of the epidermis carefully. When the first detachment of the epidermis is visible at the cut edges of skin samples, carefully separate epidermis from dermis. Place the epidermis (dermis side down) in 100-mm plastic Petri dishes and irrigate with 10 ml Medium A, including 10% FBS and antibiotics, and 500 μl Skin Dissociation System.
8. Once all pelts have been processed, use scissors to cut epidermis and disrupt β-pleated sheets in the epidermis. Transfer all media containing skin samples into a sterile beaker or container.
9. Rinse sterile stir bar in PBS and place in cell mixture and stir gently for 20-30 min.
10. Rinse woven cloth 2× in PBS and place into sterile funnel on top of sterile beaker and strain the cell mixture to remove debris (alternatively, a Nylon gauze cell strainer can be used).
11. Repeatedly aspirate and dispel the cell mixture with a sterile pipette several times to facilitate a single cell mixture.
   12. Count viable cells and plate at 10×10⁶ cells/100 mm dish.
   13. After 2½ hours, remove media and add 10 ml of complete medium B.

### Primary Culture:

14. Seed cells at 37° C. in Medium A, or Medium B, at desired densities:
   (a) Culture with feeder layer cells:
      Prepare 100 ml complete Medium A by adding 10 ml Medium A supplement into 90 ml Medium A and 100 μl Skin FibrOut™.
      On dishes that contain feeder layers, seed Keratinocytes at 2-5×10⁴ cells/cm².
   Note: Feeder layer preparation: grow 3T3 or human fibroblast cells for 3 days, followed by irradiating 3T3 cells at 30 Gy or human fibroblasts at 70 Gy.
   (b) Culture without feeder layer:
      Prepare complete Medium B by adding 10 ml Medium B supplement to 90 ml Medium B and 100 μl Skin FibrOut™.
      Culture the isolated epidermal cells (1-5×10⁴ cells/cm²) in desired flasks or dishes (collagenase pre-coated) with the Medium A, including 10% FBS, for 2-3 h, replace the medium with complete Medium B, and maintain them at high density in subcultures.
15. When cells have attached (after 1-3 d), rinse cultures extensively with culture medium to eliminate nonattached dead and differentiated cells, and continue cultivation in either Medium A or B. Stratification and slowing down of growth can be achieved by shifting the Ca²⁺ concentration in Medium B.

### Subculture:

16. Subculture as follows:
   (a) Cultures in Medium A:
      (i) Incubate in 0.05-0.1% EDTA for 5-15 min to initiate cell detachment, which is visible by the enlargement of intercellular spaces.
      (ii) Incubate in 0.1% trypsin and 1.3 mM (0.05%) EDTA at 37° C. for 5-10 min, followed by gentle pipetting, to completely detach the cells.
   (b) Cultures in Medium B:
      (i) EDTA pretreatment is not required, due to the low Ca²⁺ concentration.
      (ii) Incubate in 0.1% trypsin with 1.3 mM EDTA, as with Medium A cultures.

### Cryopreservation:

17. Cryopreservation is often necessary to maintain large quantities of cells derived from the same tissue sample; the best results are reported when cells from preconfluent primary cultures are used.
   (a) Trypsinize cells, as before, and centrifuge at 100 g for 10 min.
   (b) Resuspend cells in complete culture medium with serum and count.
   (c) Dispense aliquots of 2×10⁶ cells/ml in complete Medium A with additional 10% FBS and 10% glycerol into cryopreservation tubes.
   (d) Equilibrate at 4° C. for 1-2 h.
   (e) Freeze cells with a freezing apparatus at a cooling rate of 1° C. per min.
   (f) To recover cells:
      (i) Thaw cryotubes quickly in a 37° C. water bath.
      (ii) Dilute cells tenfold with medium.
      (iii Centrifuge cells, resuspend them at an appropriate concentration in the desired culture medium, and seed culture vessel.

Mouse cells can be grown in all three media types for several months. Mouse cells can be subcultured once or twice in Medium A and Medium B, respectively.

### Characterization

Cultured cells have to be characterized for their epidermal (epithelial) phenotype to exclude contamination by mesenchymal cells. This is best achieved using cytokeratin-specific antibodies for the epithelial cells. Contaminating endothelial cells can be identified by antibodies against CD31 or factor VIII-related antigen. Identifying fibroblasts unequivocally is difficult, because the use of antibodies against vimentin (the mesenchymal cytoskeletal element) is not specific. Keratinocytes in vitro may initiate vimentin synthesis at frequencies that depend on culture conditions. As a practical assessment for mesenchymal cell contamination, cells should be plated at clonal densities (1-5×10² cells/cm²) on feeder cells, and clone morphology should be identified at low magnification following fixation and hematoxylin and eosin (H&E) staining of 10- to 14-d cultures. A more specific and highly sensitive method to identify contaminating fibroblasts is the analysis of expression of Keratinocyte growth factor (KGF) by RT-PCR. Since this factor is produced in fibroblasts, and not in Keratinocytes, it represents a selective marker. Moreover, KGF expression is enhanced by co-cultured Keratinocytes so that a minority of contaminating fibroblasts will be detected by this assay.

### References:

1. Bickenbach, J. R., and Chism, E., “Selection and extended growth of murine epidermal stem cells in culture”, Exp. Cell Res. 244:184-195 (1998).
2. Boyce, S. T., and Ham, R. G., “Calcium-regulated differentiation of normal human epidermal kaeratinocytes in chemically defined clonal culture and serum-free serial culture”, J. Invest. Dermal. 81:33-40s (1983).
3. Dlugosz, A. A., Glick, A. B., Tennenbaum, T., Weinberg, W. C., and Yuspa, S. H., “Isolation and utilization of epidermal keratinocytes for oncogene research”, Methods in Enzymol. 254:3-20 (1995).
4. German, L., Rouabhia, M., Guignard, R., Carrier, L., Bouvard, V., and Auger, F. A. “Improvement of human keratinocyte isolation and culture using thermolysin”, Burns 19:99-104 (1993).
5. Smola, H., Thiekotter, G., and Fusenig, N. E., “Mutual induction of growth factor gene expression by epidermal-dermal cell interaction”, J. Cell Biol. 122:417-429 (1993).
6. Stark, H.-J., Baur, M., Breitkreutz, D., Mirancea, N. and Fusenig, N. E., “Organotype keratinocyte cocultures in defined medium with regular epidermal morphogenesis and differentiation”, J. Invest. Derm. 112:681-691 (1999).

### Example 3

### Growth of Rat Brain: Cerebellar Granule Cells

The following protocol is developed for the attachment and growth of normal rat brain: cerebellar granule cells using the primary cell culture system of the present invention.

### Rat Brian PrimaCell™ I: Cerebellar Granule Cells

### General Description:

Nerve cells appear to be more fastidious in their choice of substrate than most other cells. They will not survive well on untreated glass or plastic, but will demonstrate neurite outgrowth in collagen and poly-D-lysine. Neurite outgrowth is encouraged by a polypeptide nerve growth factor (NGF) and factors secreted by glial cells that are immunologically distinct from NGF. Cell proliferation has not been found in cultures of most neurons, even with cells from embryonic stages in which mitosis was apparent in vivo; however, recent studies with embryonic stem cells have shown that some neurons can be made to proliferate in vitro and re-colonize in vivo.

Cerebellar granule cells in culture provide a well characterized neuronal cell population that is suited for morphological and biochemical studies. The cells are obtained from the cerebella of 7- or 8-day-old rat (or 14 or 15-day-old mouse), and non-neuronal cells are prevented from growing by the brief addition of Brain Fibroblast Growth Inhibitors, FibrOut™, to the culture's media.

The Brain PrimaCell™ I system is suited for culturing cerebellar neurons of rat.

### The Rat Brain PrimaCell™ I System Includes:

Rat Brain Tissue Dissociation System, bTDS (2 aliquots) (0.05% Collagenase I from Clostridium Histolyticum; 1.5% Collagenase II from Clostridium Histolyticum; 0.5% Collagenase IV from Clostridium Histolyticum; 0.03% Collagenase from Clostridium Histolyticum; 3.0% Trypsin from Bovine Pancreas)
bTDS Reconstitution Buffer, (2×1 ml) (0.01% HCl; 5% BSA)
bTDS Buffer, (2×10 ml) (5.0 mM NaCl; 0.5 mM KCl; 0.15 mM CaCl₂; 0.10 mM MgCl₂; 0.10 mM MgSO₄; 1.0 mM Na₂HPO₄; 0.05 mM KH₂PO₂; 0.4 mM NaHCO₃; 5.0 mM Glucose, 5% BSA
Rat Brain Fibroblast Growth Inhibitors—FibrOut™ (1.0 ml) (5% Collagenase 4.0 nM D-valine, 1.0 nM Cis-OH-proline.)
Rat Cerebella Basal Culture Medium (500 ml) (DMEM with 30 mM Glucose, 2 mM L-Glutamine, 25 mM KCl)
Rat Cerebella Culture Medium Supplements with Serum (5×10 ml) (7 μM P-aminobenzoic acid, 15% heat-inactivated fetal calf serum, 100 μg/ml Gentamycin, 100 mU/L Insulin)
Buffer Systems for Sample and Culture Preparation (Hank's BSS with 3 g/L BSA (HBSS))

### Required Materials (but not Provided):

1. PBS (1×)

2. Water bath.

3. Pasteur pipettes

4. Culture dishes

5. 35-mm tissue culture Petri dishes

6. Scalpels, scissors, and forceps

7. Pasteur pipettes and 10-ml pipettes

8. Test tubes, 12 and 50 ml

### Siliconization of Pasteur Pipettes:

Dilute the siliconizing solution, such as Apuasil solution (10% stock), to 1% in sterilized water.
Dip the pipettes into the solution, or flush out the insides of the pipettes.
Air dry the pipettes for 24 h, or dry for several minutes at 100° C.
Sterilize the pipettes by dry heat.

### Poly-L-Lysine Treatment of Culture Dishes:

Add 1 ml of poly-L-lysine solution (10 mg/ml), diluted to 10 mg/L before use, to each of the 35-mm Petri dishes.
Remove the poly-L-lysine solution after 10-15 min, and add 1-15 ml of culture medium.
Place the culture dishes in the incubator (minimum 2 h) until the cells are to be seeded.

### Principle

The cerebella from four to eight neonatal rats or mice are cut into small cubes and incubated with the Brain Tissue Dissociation System (bTDS, 10 ml diluted bTDS solution per mg tissue sample, 1:10 dilution from the stock) for 30 min at 37° C. The cell suspension is seeded on poly-L-lysine-coated culture wells or flasks.

### Protocol

### Specimens:

1. Rat Brain: Newborn (prefer 14-16 days) mouse or rat (7-8 days) yields a large number of cells (5-10×10⁶), with a 30-40% plating efficiency. Rats are sacrificed by CO₂ narcosis or a method that is approved by user's institution. Carefully remove the skin. Dissect out the cerebella aseptically and place them in 1×PBS buffer.
2. Mince the tissue with scalpels into small cubes, approximately 0.5 mm³.
3. Transfer the minced tissue to test tubes (12 ml) and wash the tissue three times with 1× PBS. Allow the tissue to settle to the bottom of the tubes between each washing.
4. Reconstitute bTDS stock solution by adding 1 ml bTDS reconstitution buffer per tube of lyophilized enzyme mixtures. Add the diluted Brain Tissue Dissociation System (1:10 in bTDS Buffer) to the tissue. We recommend using 10 ml diluted bTDS solution per mg tissue sample, and incubating the tube in a water bath for 30 min at 37° C.
5. Transfer the dissociated tissue to a 50-ml centrifuge tube, and add 20 ml of the complete culture medium containing the growth supplements with serum.
6. Disaggregate the tissue by repeated aspiration and expelling through a siliconized Pasteur pipette, until a single-cell suspension is obtained.
7. Let the cell suspension stay in the centrifuge tube for 3-5 min, allowing small clumps of tissue to settle to the bottom of the tube. Remove these clumps with a Pasteur pipette.
8. Centrifuge the single-cell suspension at 200 g for 5 min, and aspirate off the supernatant.
9. Resuspend the pellet in complete culture medium containing cerebella culture medium supplements with serum, and seed the cells at a concentration of 2.5-3.0×10⁶ cells/dish.
10. After 2-4 d (best results usually are obtained after 2 d), culture cells with medium containing the Rat Brain Fibroblast Growth Inhibitors, FibrOut™ (1:500 dilution) for 24 h.
11. Change to complete culture media without FibrOut™.

### Characterization

Neurons can be identified immunologically by using neuron-specific enolase antibodies or by using tetanus toxin as a neuronal marker. Astrocyte contamination can be quantified by using glial fibrillary acidic protein as a marker.

### Variations

A single-cell suspension can be obtained by mechanical sieving through nylon meshes of decreasing diameter or by sequential treatment of Tissue Dissociation System solution (i.e. a 3-5 min treatment).

### References:

1. Drejer J, Larsson O M, Schousboe A, “Characterization of uptake and release processes for D- and L-aspartate in primary cultures of astrocytes and cerebellar granule cells”, Neurochem Res, 8(2):231-43 (1983).
2. Kingsbury A E, Gallo V, Woodhams P L, Balazs R, “Survival, morphology and adhesion properties of cerebellar interneurones cultured in chemically defined and serum-supplemented medium”, Brain Res, 349(1-2):17-25 (1985).
3. Thomson J A, Itskovitz-Eldor J, Shapiro S S, Waknitz M A, Swiergiel J J, Marshall V S, Jones J M, “Embryonic stem cell lines derived from human blastocysts”, Science, 282(5391): 1145-7 (1998).
4. Levi-Montalcini R, Calissano P, “The nerve-growth factor”, Sci Am, 240(6):68-77 (1979).
5. Marchionni M A, Goodearl A D, Chen M S, Bermingham-McDonogh O, Kirk C, Hendricks M, Danehy F, Misumi D, Sudhalter J, Kobayashi K, et al., “Glial growth factors are alternatively spliced erbB2 ligands expressed in the nervous system”, Nature, 362(6418):312-8 (1993).

### Example 4

### Growth of Mouse Skin: Melanocytes

The following protocol is developed for the attachment and growth of normal mouse melanocytes using the primary cell culture system of the present invention.

### Mouse Skin PrimaCell™ I: Melanocytes

### I. General Description:

This protocol is developed for attachment and growth of normal mouse skin melanocytes from newborn or adult mouse skin with the Mouse Skin PrimaCell™ I system. This system provides the tissue dissociation system, Skin OptiTDS™, that routinely yields 4-7 times more cells than most of the tissue dissociation protocols published in the literature. In addition, this system ensures a high viability of the target cells with improved gradient contained in the culture medium. With CHI's proprietary fibroblast inhibitory system, FibrOut™, cells grow with contamination of a minimized amount of non-epithelial cells.

The preparation of tissue specimens for cell culture is usually started within 1-2 h of removal from mice. If this is impossible, fine cutting of the tissue into small pieces (2×2 mm) with scalpels and storage overnight at 4° C. in washing medium (see below) can also prove successful.

The Mouse Skin PrimaCell™ I system applies to all types of skin samples from mice at all ages, although newborn mice are recommended for yielding the maximum amount of viable target cells. Skin samples containing pathological organisms (viruses, parasites, etc.) or tumors may not be suitable for this system.

### 1.1 Components of Mouse Skin PrimaCell™ I System

Skin Tissue Dissociation System, Skin OptiTDS™, (2 aliquots)—A mixture of 5% collagenase I, 0.3% collagenase II, 1% collagenase IV, 1% dispase and 1.5% trypsin.
Skin OptiTDS™ Reconstitution Buffer, (2×1 ml). (0.01% HCl; 5% BSA.
Skin OptiTDS™ Digestion Buffer, (2×10 ml). (10 mM NaCl; 0.5 mM KCl; 0.15 mM CaCl₂; 0.50 mM MgCl₂; 0.50 mM MgSO₄; 1.0 mM Na₂HPO₄; 0.05 mM KH₂PO₂; 0.4 mM NaHCO₃; 3.0 mM Glucose)
Skin Tissue Washing Medium, (5×10 ml)—Basal Skin PrimaCell™ I Culture Medium with 5% FBS, 200 u/ml of penicillin, 200 μg/ml of streptomycin, and 50 μg/ml of gentamycin.
Mouse Skin Fibroblast Growth Inhibitors, Skin FibrOut™ (5×200 μl)—A mixture of 1 nM cis-OH-proline, 0.1% collagenase, 3 nM D-valine, and 5% formulated serum substitutes (Bovine Growth Serum, Cat No. SH30541, HyClone, UT)
Mouse Skin PrimaCell™ I Basal Culture Medium, (5×100 ml)—Modified formulation based on medium 199 and Weymouth medium by mixing the two according to a ratio of 1:9, respectively.
Mouse Skin PrimaCell™ I Medium Supplements, (5×1.0 ml)—A mixture of 10 nM basic fibroblast growth factor, 0.1 nM cholera toxin, 0.5 μg/ml hydrocortisone, 5 μg/ml Insulin, 20 μg/ml transferrin, and 0.1 ng/ml epidermal growth factor.
Mouse Skin PrimaCell™ I Serum, (1×50 ml)—Highly purified and 24 hour-10% charcoal-dextran-stripped Fetal-bovine serum.

### 1.2 Required Materials (not Provided)

1. 70% sterile ethanol

2. PBS

3. Pasteur pipettes

4. Collagen I-coated Culture dishes

5. Scalpels, scissors, and forceps

6. Pasteur pipettes and 10-ml pipettes

7. Test tubes, 12 and 50 ml

8. Nylon gauze cell strainer

### II. Procedures

### 2.1 Material Preparation

Materials used in this experiment should be sterile or autoclaved to prevent contamination. To enhance cell attachment to the culture dishes, collagen I-coated plates (Corning, N.Y.) are recommended.

### 2.2 Surgical Specimens

1. Newborn (prefer 1-2 days) mouse epidermis is recommended for yielding a large number of cells (5-10×10⁶/epidermis), with a 30-40% plating efficiency. Mice are sacrificed by CO₂ narcosis 1-4 days postpartum (prior to the appearance of hair). Using an aseptic technique, limbs and tails are amputated, a longitudinal incision is made from tail to snout, and skin is peeled off the carcass using forceps.
2. Incubate skin biopsies for up to 10 min in Skin Tissue Washing Medium to prevent infection. (This procedure should not affect Melanocyte viability.)
3. Incubate skin biopsies in 10 ml 70% sterile ethanol for 2 min.
4. Rinse tissue twice in Skin Tissue Washing Medium for 10 min each and kept on ice.

### 2.3 Tissue Preparation and Dissociation

### 2.3.1 Mouse Skin OptiTDS™

In the primary cell culture, there are several important factors that can affect the yield and viability of cells, including type of tissues, origin of species, age of the animal used, enzymes, culture media and growth supplements. The Mouse Skin Tissue Dissociation System, OptiTDS™, is suited for optimal dissociation of normal adult and newborn skin biopsy samples to yield the maximum number of primary cells of colonic tissues.

### 2.3.2 Enzyme Compositions

Trypsin: from Bovine Pancreas

Dispase: from Bacillus polymyxa

Collagenase I: from Clostridium Histolyticum

Collagenase II: from Clostridium Histolyticum

Collagenase IV: from Clostridium Histolyticum

### 2.3.3 System Components

Skin Tissue Dissociation System, OptiTDS™, 2 vials.

Skin OptiTDS™ Reconstitution Buffer, (2×1 ml).

Skin OptiTDS™ Digestion Buffer, (2×9 ml).

### 2.3.4 Procedures for Tissue Preparation and Dissociation

1. Prepare fresh enzyme working solutions: to each vial of Skin Tissue Dissociation System, OptiTDS™, add 1.0 ml of the Skin OptiTDS™ Reconstitution Buffer. Mix well.
2. Add 1.0 ml of the fresh enzyme working solution to one vial of Skin OptiTDS™ Digestion Buffer (9.0 ml). Warm the diluted Skin OptiTDS™ working solution at 37° C. for 10 min prior to use. For optimal results, we recommend using 2-3 g tissue samples per 10 ml of diluted Skin OptiTDS™ working solution.
3. Mince pre-washed tissue into pieces approximately 0.2-0.5 mm² in diameter.
4. Incubate tissue with Skin Dissociation System by one of the following steps:
   (a) Rapid dissociation: Float skin samples in Skin Dissociation System solution for 2-3 h at 37° C. This works particularly well with skin of full-thickness.
   (b) Slow dissociation: Float the samples on ice-cold Skin Dissociation System at 4° C. for 15-24 h.
5. Monitor the separation of the epidermis carefully. When the first detachment of the epidermis is visible at the cut edges of skin samples, carefully separate epidermis from dermis. Place the epidermis (dermis side down) in 100-mm plastic Petri dishes and irrigate with 10 ml fresh Skin OptiTDS™ solution.
6. Once all pelts have been processed, use scissors to cut epidermis and disrupt β-pleated sheets in the epidermis. Transfer all solution containing skin samples into a sterile beaker or container.
7. Rinse a sterile stir bar in PBS and place in cell mixture and stir gently for 20-30 min.
8. Rinse woven cloth 2× in PBS and place into sterile funnel on top of sterile beaker and strain the cell mixture to remove debris (alternatively, a Nylon gauze cell strainer can be used).
9. Pass the cell mixture with a sterile pipette several times to facilitate a single cell mixture and pass through the Nylon gauze cell strainer.
10. Collect cells by centrifugation at 100 g, washing cells with 10 ml PBS or washing media twice. At the end of the washing process, collect the cells and dilute in 0.5-1.0 ml complete culture medium.
11. Count viable cells and plate at 1.0×10⁶/100 mm dish.
12. Seed the cells at 37° C. in Complete Mouse Skin PrimaCell™ I Culture Medium at desired densities.

### 2.3.5 Storage:

Tissue dissociation systems should be reconstituted before use, and the solution can only be stored for 2-4 days at 4° C. For long-term use, it should be aliquotted and stored at −20° C. Avoid repeated freeze-thaw cycles.

### 2.4 Culture of Primary Mouse Keratinocytes

### 2.4.1 Medium Preparation.

Thaw out the Mouse Skin PrimaCell™ Medium Supplements and Mouse Skin PrimaCell™ I Serum on ice. To every 100 ml Mouse Skin PrimaCell™ I Basal Culture Medium, add one vial of Mouse Skin PrimaCell™ I Medium Supplements, 10 ml Mouse Skin PrimaCell™ I Serum, and one vial of Mouse Skin Fibroblast Growth Inhibitors, Skin FibrOut™. Mix thoroughly and warm the complete medium in a 37° C. water bath for 10 min prior use.

### 2.4.2 Primary Culture Conditions.

Epithelial cells derived from tissue specimens contain both Keratinocytes and Melanocytes. Seed cells onto T-25 flasks coated with collagen type I in a 5%-CO₂ incubator with 4 ml of complete culture medium. Change the culture medium twice weekly. The cultures will contain primary Keratinocytes with scattered melanocytes. Keratinocyte proliferation should cease within several days, and colonies should begin to detach during the second week. By the end of the third week, only melanocytes should remain. In most cases, cultures attain near confluence and are ready to passage within 2-4 weeks.

### 2.4.3 Subculture and Propagation

1. Gently rinse the culture dish twice with 0.02% (0.7 mM) EDTA.
2. Add 3 ml of 0.25% trypsin/0.1% (2.5 mM) EDTA and incubate at 37° C. Examine the dish under phase microscopy every 5 min to detect cell detachment.
3. When most cells have detached, add 12 ml complete melanocyte growth medium to inactive the trypsin.
4. Pipette the contents of the dish to ensure complete melanocyte detachment.
5. Aspirate and centrifuge the cells for 5 min at 350 g.
6. Aspirate the supernatant, resuspend the cells in a complete growth medium, and re-plate at 2-4×10⁴ cells per 100-mm dish.
7. Re-feed the culture twice a week with complete melanocyte growth medium.

### III Cryopreservation

Cryopreservation is often necessary to maintain large quantities of cells derived from the same tissue sample; the best results are reported when cells from pre-confluent primary cultures are used.
1. Detach cells as for subculture, and centrifuge at 100 g for 10 min.
2. Re-suspend cells in complete culture medium with serum and count.
3. Dispense aliquots of 2×10⁶ cells/ml in complete growth medium with additional 10% FBS and 10% glycerol into cryopreservation tubes.
4. Equilibrate at 4° C. for 1-2 h.
5. Freeze cells with a freezing apparatus at a cooling rate of 1° C. per min.
6. To recover cells:
   (i) Thaw cryotubes quickly in a 37° C. water bath.
   (ii) Dilute cells tenfold with medium.
   (iii) Centrifuge cells, resuspend them at an appropriate concentration in the desired culture medium, and seed culture vessel.

### Mouse cells can be grown in all media for 4-7 weeks and can be subcultured only 4-5 times.

### IV Fibroblast Contamination

There are several techniques published in the literature to deal with fibroblast contamination during melanocyte primary cell culture. (1) Physically removing a well-isolated fibroblast colony by scraping it with a sterile blunt instrument (e.g., a cell scraper) Care has to be taken to wash the culture up to six times to remove any fibroblasts that have detached in order to prevent them from reseeding and reattaching to the flask. (2) Differential trypsinization can be attempted with the carcinomas. (3) Dispase preferentially (but not exclusively) removes the epithelium during passaging and leaves behind most of the fibroblastic cells attached to the culture vessel. During subculture, cells that have been removed with dispase can be pre-incubated in plastic petri dishes for 2-6 h to allow the preferential attachment of any fibroblasts that may have been removed together with the epithelium. This technique takes advantage of the fact that fibroblasts, in general, attach much more quickly to plastic than do clumps of melanocytes, so that a partial purification step is possible. (4) Reduce the concentration of serum to about 2.5-5% if there are heavy concentrations of fibroblastic cells. It is worth remembering that normal fibroblasts have a finite growth span in vitro and that using any or all of the preceding techniques will eventually push the cells through so many divisions that any fibroblasts will senesce.

The Mouse Skin PrimaCell™ I system includes a fibroblast elimination system, the Mouse Skin Fibroblast Growth Inhibitors, Skin FibrOut™. It contains a mixture of cis-OH-proline, collagenase, D-valine, and formulated serum substitutes. This system can effectively eliminate skin fibroblast contamination while having no affect on the behavior of melanocytes.

### V Confirmation of Melanocytic Identity

Melanocyte cultures may be contaminated initially with keratinocytes and at any time by dermal fibroblasts. Both forms of contamination are rare in cultures established and maintained by an experienced technician or investigator but are common problems for the novice. The cultured cells can be confirmed to be melanocytes with moderate certainty by frequent examination of the culture under phase microscopy, assuming that the examiner is familiar with the respective cell morphologies. More definitive identification is provided by electron microscopic examination, DOPA staining, or immunofluorescent staining with Mel 5 antibody, directed against tyrosinase-related protein-1.

### VI References:

1. Naeyaert J M, Eller M, Gordon P R, Park H Y, Gilchrest B A, “Pigment content of cultured human melanocytes does not correlate with tyrosinase message level”, Br J Dermatol, October; 125(4):297-303 (1991).
2. Gilchrest B A, Vrabel M A, Flynn E, Szabo G, “Selective cultivation of human melanocytes from newborn and adult epidermis”, J Invest Dermatol, November; 83(5):370-6 (1984).
3. Wilkins L, Gilchrest B A, Szabo G, Weinstein R, Maciag T, “The stimulation of normal human melanocyte proliferation in vitro by melanocyte growth factor from bovine brain”, J Cell Physiol, March; 122(3):350-61 (1985).
4. Naeyaert J M, Eller M, Gordon P R, Park H Y, Gilchrest B A, “Pigment content of cultured human melanocytes does not correlate with tyrosinase message level”, Br J Dermatol, October; 125(4):297-303 (1991).
5. Park H Y, Gilchrest B A, “Protein kinase C: biochemical characteristics and role in melanocyte biology”, J Dermal Sci, December; 6(3):185-93 (Review) (1993).

### 6. Guyonneau L, Murisier F, Rossier A, Moulin A, Beermann F, “Melanocytes and pigmentation are affected in dopachrome tautomerase knockout mice”, Mol Cell Biol. April; 24(8):3396-403 (2004).

7. Hirobe T, Furuya R, Ifuku O, Osawa M, Nishikawa S, “Granulocyte-macrophage colony-stimulating factor is a keratinocyte-derived factor involved in regulating the proliferation and differentiation of neonatal mouse epidermal melanocytes in culture”, Exp Cell Res, July 15; 297(2):593-606 (2004).
8. Hirobe T, Osawa M, Nishikawa S, “Hepatocyte growth factor controls the proliferation of cultured epidermal melanoblasts and melanocytes from newborn mice”, Pigment Cell Res, February; 17(1):51-61 (2004).
9. Hirobe T, “Endothelins are involved in regulating the proliferation and differentiation of mouse epidermal melanocytes in serum-free primary culture”, J Investig Dermal Symp Proc, November; 6(1):25-31 (2001).

### Example 5

### Growth of Mouse Kidney Epithelium

This protocol is developed for attachment and growth of normal mouse kidney epithelial cells from newborn or adult mouse using the primary cell culture system of the present invention.

### Mouse Kidney PrimaCell™: Kidney Epithelium

### I. General Description

This protocol is developed for attachment and growth of normal mouse kidney epithelial cells from newborn or adult mouse kidney with the Mouse Kidney PrimaCell™ system. This system provides an optimal tissue dissociation system, Kidney OptiTDS™, that routinely yields 4-7 times more cells than most of the tissue dissociation protocols published in the literature. In addition, this system ensures a high viability of the target cells with improved gradient contained in the culture medium. With CHI's proprietary fibroblast inhibitory system, FibrOut™, cells grow with contamination of a minimized amount of non-epithelial cells.

The preparation of tissue specimens for cell culture is usually started within 1-2 h of removal from mice. If this is impossible, fine cutting of the tissue into small pieces (1 mm cubes) with scalpels and storage overnight at 4° C. in washing medium (see below) can also prove successful. With this system, large numbers of cells can be harvested, making it practical to establish multiple replicate primary cultures or to propagate cells for frozen storage.

The Mouse Kidney PrimaCell™ system applies to kidney samples from mice of all ages, although 2 to 5 month old mice are recommended for yielding the maximum amount of viable target cells. Kidney samples containing pathological organisms (viruses, parasites, etc.) or tumors may not be suitable for this system.

### 1.1 Components of the Mouse Kidney PrimaCell™ System

Kidney Tissue Dissociation System, Kidney OptiTDS™, (2 vials)—A mixture of 0.1% collagenase I, 0.5% collagenase, 0.2% DNase I, 1% dispase and 2% trypsin.
Kidney OptiTDS™ Reconstitution Buffer, (2×1 ml).—0.01% HCl; 5% BSA
Kidney OptiTDS™ Digestion Buffer, (2×9 ml)—7.0 mM NaCl; 0.5 mM KCl; 0.15 mM CaCl₂; 0.10 mM MgCl₂; 0.10 mM MgSO₄; 2.0 mM Na₂HPO₄; 0.05 mM KH₂PO₂; 0.4 mM NaHCO₃; 1.0 mM Glucose.
Kidney Tissue Washing Medium, (2×100 ml)—Basal Kidney PrimaCell™ Culture Medium with 5% FBS, 200 u/ml of penicillin, 200 μg/ml of streptomycin, and 50 μg/ml of gentamycin.
Kidney Fibroblast Growth Inhibitors, Kidney FibrOut™ (5×200 μl)—A mixture of 1.0 nM cis-OH-proline, 3% collagenase, 5.0 μM D-valine, and 5% formulated serum substitutes (Bovine Growth Serum, Cat No. SH30541, HyClone, UT) 1.5 μM Fluvastatin; and 1.0 M sodium ethylmercurithiosalicylate.
Mouse Kidney PrimaCell™ Basal Culture Medium, (5×100 ml)—Modified formulation based on medium DMEM and F-12 by adding 0.2 mM NaCl, 3 mM CaCl₂, 1.0 mM Na₂HPO₄, and 0.5 mM NaH₂PO₄ to a mixture of equal volume of DMEM and F-12.
Mouse Kidney PrimaCell™ Medium Serum, (1×50 ml)—Highly purified and 10% charcoal-dextran-stripped Fetal-bovine serum.
Mouse Kidney PrimaCell™ Medium Supplements, (5 vials)—A mixture of 5 μg/ml insulin, 2 nM sodium selenite, 25 μg/ml transferrin, 2 nM 3,3′5-triiodothyronine, 10% highly purified bovine serum albumin, and 2.0 mM glutamine, 1.0 nM dexamethasone, antibiotics ((100 U/ml) penicillin, (50 μg/ml) streptomycin)

### 1.2 Required Materials but not Provided

1. Centrifuge tube, 50 ml

2. Nylon gauze cell strainer (BD Bioscience)

3. Petri dishes, collagenase-I coated, 100 mm (Corning, N.Y.)

4. Scalpels, curved forceps

5. 70% ethanol, sterile

6. 0.05% EDTA (pH 7.4), sterile

7. 0.25% trypsin/0.1% (2.5 mM) EDTA, sterile

8. PBSA (PBS containing 10% BSA), sterile.

### II. Procedures

### 2.1 Procedure Outline and Material Preparation

Tissue fragments are excised from the outer cortex of the kidney, minced, washed, and incubated (with agitation) in Kidney OptiTDS™ solution, which is a mixture of several tissue dissociation enzymes. The Kidney Dissociation System is developed for optimal Kidney tissue dissociation. The isolated epithelium is further dispersed by additional incubation in medium containing the Kidney Dissociation System, or mechanically, by pipetting. After which, it is filtered through nylon gauze and propagated in a complete kidney cell growth medium.

All materials and equipment used in this experiment should be sterilized and rinsed with PBSA prior to use.

### 2.2 Surgical Specimens

1. Mouse Kidney: Two mice (males and/or females from 2 to 5 months old) at a time were sacrificed by halothane inhalation. The mice were doused with 70% ethanol to minimize contamination of the primary cultures. Kidneys were removed using scissors and forceps soaked in 70% ethanol, and as each organ was removed, it was immediately placed in a 100-mm tissue culture dish containing 10 ml sterile PBS (each organ type was placed in a separate dish). After all organs were removed, they were transferred to fresh 100-mm dishes containing 10 ml of sterile PBS. Kidneys were minced into 1-mm cubes using razor blades dipped in 70% ethanol. The minced tissues were transferred into sterile 15-ml conical tubes containing sterile PBS. After allowing the minced tissue pieces to settle, the PBS was aspirated, and the tissues were washed once more with sterile PBS.
2. Incubate kidney biopsies sequentially in 10 ml 70% ethanol for 2 min, in 10 ml PBSA for 2 min, and in 20 ml Kidney Tissue Washing Medium for 10 min, to prevent infection. (This procedure is not affecting cell viability.)
3. Keep on ice until the tissue dissociation procedure.

### 2.3 Tissue Preparation and Dissociation

### 2.3.1 Mouse Kidney OptiTDS™

In the primary cell culture, there are several important factors that can affect the yield and viability of cells, including type of tissues, origin of species, age of the animal used, enzymes, culture media and growth supplements. The Mouse Kidney Tissue Dissociation System, OptiTDS™, is suited for optimal dissociation of normal adult and newborn kidney biopsy samples to yield the maximum number primary cells of colonic tissues.

### 2.3.2 Enzyme Compositions

2.0% Trypsin: from Bovine Pancreas

1.0% Dispase: from Bacillus polymyxa

0.1% Collagenase I: from Clostridium Histolyticum

0.5% Collagenase: from Clostridium Histolyticum

0.2% DNase I: from Clostridium Histolyticum

### 2.3.3 System Components

Kidney Tissue Dissociation System, OptiTDS™, 2 vials.

Kidney OptiTDS™ Reconstitution Buffer, (2×1 ml).

Kidney OptiTDS™ Digestion Buffer, (2×9 ml).

### 2.3.4 Procedures for Tissue Preparation and Dissociation

4. Prepare fresh enzyme working solutions: to each vial of Kidney Tissue Dissociation System, OptiTDS™, add 1.0 ml of the Kidney OptiTDS™ Reconstitution Buffer. Mix well.
5. Add 1.0 ml of the fresh enzyme working solution to one vial of Kidney OptiTDS™ Digestion Buffer (9.0 ml). Warm the diluted enzyme working solution at 37° C. for 10 min prior to use. For optimal results, we recommend using 5-7 mg tissue samples per 10 ml diluted enzyme working solutions.
6. Rinse surgical instruments in sterile PBS.
7. Mince pre-washed tissue into approximately 1 mm cube pieces with scalpel and forceps.
8. Incubate minced tissue with the Kidney Dissociation System by one of the following steps:
   a. Rapid dissociation: Incubate kidney samples, with rocking, in the Kidney Dissociation System solution for 2-3 h at 37° C. This works particularly well with full-thickness kidney.
   b. Slow dissociation: Incubate the samples, with rocking, in ice-cold Kidney Dissociation System at 4° C. for 15-24 h.
9. Rinse woven cloth 2× in PBS and place into a sterile funnel on top of a sterile beaker, and strain the cell mixture to remove debris (alternatively, a Nylon gauze cell strainer can be used).
10. Repeatedly aspirate and expel the cell mixture with a sterile pipette several times to facilitate a single cell mixture, and collect the cells by passing the mixture through the woven cloth or Nylon gauze cell strainer
11. The filtrate, which contained single cells dissociated from the tissue specimens, were collected into sterile 15-ml conical tubes by centrifugation at 800 g for 5 min. The resulting pellet containing the primary culture cells was re-suspended in 5 ml of complete culture medium containing 10% fetal bovine serum and growth supplements. Save the remaining tissues contained in the debris for repeating the tissue dissociation process in step 9.
12. For the remaining pieces of tissue, repeat the tissue dissociation process with the saved Kidney Dissociation working solutions from step 8 to yield additional cells. This process can be repeated up to 3 times if it is necessary without changing the enzyme working solution. In general, kidneys from each 5-6 month old mouse should produce six to eight 60-mm dishes of primary culture cells.
13. Count viable cells and plate at a density of 1×10⁵ cells per cm².

### 2.3.5 Storage:

The tissue dissociation system should be reconstituted before use and can only be stored for 2-4 days at 4° C. For long-term use, it should be aliquoted and stored at −20° C. Avoid repeated freeze-thaw cycles.

### 2.4 Culture of Primary Mouse Kidney Epithelium

### 2.4.1 Medium Preparation.

Thaw out the Mouse Kidney PrimaCell™ Basal Culture Medium, Mouse Kidney PrimaCell™ Medium Serum, and Mouse Kidney PrimaCell™ Medium Supplements on ice. For every 100 ml Mouse Kidney PrimaCell™ Basal Culture Medium, add 10 ml Mouse Kidney PrimaCell™ Medium Serum, one vial of Mouse Kidney PrimaCell™ Medium Supplements, and one vial of Mouse Kidney Fibroblast Growth Inhibitors, Kidney FibrOut™. Mix thoroughly and warm the complete medium in a 37° C. water bath for 10 min prior to use.

### 2.4.2 Primary Cell Culture.

The primary cultures will be placed in a 37° C., 5% CO₂ humidified incubator for 24 hr to allow cells to adhere. After 24 hr, the cells will be washed twice with sterile PBS to remove non-adherent cells (such as blood cells, etc.) and tissue fragments. Return cells to the 37° C. incubator in the medium described above for another 2-3 days (until they reach 60-80% confluences). After this time, cells from the same organ are trypsinized, combined, and split into the number of 60-mm tissue-culture dishes required for a single experiment (usually 17-24 dishes). Cells usually require an additional 3-4 days to grow to the appropriate density for an experiment.

### 2.4.3 Subculture and Propagation

1. Gently rinse the culture dish twice with 0.02% (0.7 mM) EDTA.
2. Add 3 ml of 0.25% trypsin/0.1% (2.5 mM) EDTA and incubate at 37° C. Examine the dish under phase microscopy every 5 min to detect cell detachment.
3. When most of the cells have detached, add 10 ml complete Kidney cell growth medium to terminate the trypsin activity.
4. Pipette the contents of the dish to ensure complete Kidney cell detachment.
5. Aspirate and centrifuge the cells for 5 min at 350 g.
6. Aspirate the supernatant, resuspend the cells in a complete growth medium, and re-plate at a density of 1×10⁵ cells per cm².
7. Re-feed the culture twice a week with complete Kidney growth medium.

### III Cryopreservation

Cryopreservation is often necessary to maintain large quantities of cells derived from the same tissue sample; the best results are reported when cells from pre-confluent primary cultures are used.
(a) Trypsinize the cells as before and centrifuge at 100 g for 10 min.
(b) Resuspend cells in complete culture medium with serum, and count.
(c) Dispense aliquots of 2×10⁶ cells/ml in complete medium with additional 10% FBS and 10% glycerol into cryopreservation tubes.
(d) Equilibrate at 4° C. for 1-2 h.
(e) Freeze cells with a freezing apparatus at a cooling rate of 1° C. per min.
(f) To recover cells:
   (i) Thaw cryotubes quickly in a 37° C. water bath.
   (ii) Dilute cells tenfold with medium.
   (iii) Centrifuge cells and resuspend them at an appropriate concentration in the desired culture medium, and seed culture vessel.

### Mouse cells can be grown for several weeks and can be subcultured only 4-6 passages in complete growth medium.

### IV Fibroblast Contamination

There are several techniques published in the literature to deal with fibroblast contamination during kidney primary cell culture. (1) Physically removing a well-isolated fibroblast colony by scraping it with a sterile blunt instrument (e.g., a cell scraper) Care has to be taken to wash the culture up to six times to remove any fibroblasts that have detached in order to prevent them from reseeding and reattaching to the flask. (2) Differential trypsinization can be attempted with the carcinomas. (3) Dispase preferentially (but not exclusively) removes the epithelium during passaging and leaves behind most of the fibroblastic cells attached to the culture vessel. During subculture, cells that have been removed with dispase can be pre-incubated in plastic petri dishes for 2-6 h to allow the preferential attachment of any fibroblasts that may have been removed together with the epithelium. This technique takes advantage of the fact that fibroblasts, in general, attach much more quickly to plastic than do clumps of melanocytes, so that a partial purification step is possible. (4) Reduce the concentration of serum to about 2.5-5% if there are heavy concentrations of fibroblastic cells. It is worth remembering that normal fibroblasts have a finite growth span in vitro and that using any or all of the preceding techniques will eventually push the cells through so many divisions that any fibroblasts will senesce.

The Mouse Kidney PrimaCell™ I system includes the Mouse Kidney Fibroblast Growth Inhibitors, Kidney FibrOut™. It contains a mixture of cis-OH-proline, collagenase, D-valine, and formulated serum substitutes. This system can effectively eliminate kidney fibroblast contamination while having no affect on the behavior of kidney epithelial cells.

### V References:

1. Kempson S A, Ying A L, McAteer J A, Murer H, “Endocytosis and Na+/solute cotransport in renal epithelial cells”, J Biol Chem, November 5; 264(31):18451-6 (1989).
2. Andreoli S P, McAteer J A, “Reactive oxygen molecule-mediated injury in endothelial and renal tubular epithelial cells in vitro”, Kidney Int, November; 38(5):785-94 (1990).
3. Yusufi A N, Szczepanska-Konkel M, Kempson S A, McAteer J A, Dousa T P, “Inhibition of human renal epithelial Na+/Pi cotransport by phosphonoformic acid”, Biochem Biophys Res Commun, September 14; 139(2):679-86 (1986).
4. Bayley S A, Stones A J, Smith C G, “Immortalization of mouse kidney by transfection with polyomavirus large T gene”, Exp Cell Res, July; 177(1):232-6 (1988).
5. Fukase M, Birge S J Jr, Rifas L, Avioli L V, Chase L R, “Regulation of 25 hydroxyvitamin D31-hydroxylase in serum-free monolayer culture of mouse kidney”, Endocrinology, March; 110(3):1073-5 (1982).
6. Alenghat F J, Nauli S M, Kolb R, Zhou J, Ingber D E, “Global cytoskeletal control of mechanotransduction in kidney epithelial cells”, Exp Cell Res, November 15; 301(1):23-30 (2004).
7. Akis N, Madaio M P, “Isolation, culture, and characterization of endothelial cells from mouse glomeruli”, Kidney Int, June; 65(6):2223-7 (2004).

### Example 6

### Culture of Human Colonic Epithelial Cells

This protocol is developed for attachment and growth of human colonic epithelial cells using the primary cell culture system of the present invention.

### Human Colon PrimaCell™: Colorectal Epithelium

### I. General Description:

This protocol is developed for attachment and growth of normal human colonic epithelial cells from 1-3 mm³ biopsies with the Human Colon PrimaCell™ system. This system provides an optimal tissue dissociation system, Colon OptiTDS™, that routinely yields 4-7 times more cells than most of the tissue dissociation protocols published in the literature. In addition, this system ensures a high viability of target cells with improved gradient contained in the culture medium. With CHI's proprietary fibroblast inhibitory system, Colon FibrOut™, cells grow with minimized contamination of the non-epithelial cells.

The preparation of tissue specimens for cell culture is usually started within 1-2 h of removal from the patient. If this is impossible, fine cutting of the tissue into small pieces (1-2 mm) with scalpels and storage overnight at 4° C. in washing medium (see below) can also prove successful.

The Human Colon PrimaCell™ system applies to all types of normal adult human biopsy samples. Biopsy samples containing pathological organisms (viruses, parasites, etc.) or tumors may not be suitable for this system.

### 1.1 Components of Human Colon PrimaCell™ System

Colonic Tissue Dissociation System, Colon OptiTDS™, (2 aliquots)—A mixture of 3.0% collagenase I, 0.5% collagenase III, 0.5% collagenase IV, 0.01% collagenase, and 1.0% trypsin
Colon OptiTDS™ Reconstitution Buffer, (2×1 ml)—0.01% HCl; 5% BSA
Colon OptiTDS™ Digestion Buffer, (2×10 ml)—5.0 mM NaCl; 0.7 mM KCl; 0.15 mM CaCl₂; 0.10 mM MgCl₂; 0.10 mM MgSO₄; 1.0 mM Na₂HPO₄; 0.05 mM NaH₂PO₄; 0.05 mM KH₂PO₂; 0.4 mM NaHCO₃; 1.0 mM Glucose; 0.02 mM Phenol red.
Colonic Tissue Washing Medium, (5×10 ml)—Basal Colon PrimaCell™ Culture Medium with 5% FBS, 200 u/ml of penicillin, 200 μg/ml of streptomycin, and 50 μg/ml of gentamycin.
Human Colon Fibroblast Growth Inhibitors, Colon FibrOut™ (5×200 μl)—A mixture of 2% anti-Thy-1 monoclonal antibody, 15 μM toxin ricin, and 5% formulated serum substitutes (Bovine Growth Serum, Cat No. SH30541, HyClone, UT) in PBS buffer.
Human Colon PrimaCell™ Basal Culture Medium, (5×100 ml)—Modified formulation based on NCTC 168 and Weymouth medium by mixing equal volume of NVTC 168 and Weymouth Medium.
Human Colon PrimaCell™ Medium Supplements, (5×1.0 ml)—A mixture of 2.0 nM ethanolamine, 1.0 nM phosphoethanolamine, 1.0 μg/ml hydrocortisone, 0.1% ascorbic acid, 25 μg/ml transferrin, 0.5 U/ml insulin, 0.2 ng/ml epidermal growth factor, 5 ng/ml pentagastrin, and 1.0 nM deoxycholic acid.
Human Colon PrimaCell™ Serum, (1×50 ml)—Heat-inactivated and 10 hour-10% charcoal-dextran-stripped Fetal-bovine serum.
Coating Solution, (5×10 ml)—Basal growth medium containing 10 μg/ml BSA.

### 1.2 Required Materials (but not Provided)

1. Hank's balanced salt solution (HBSS)

2. Dispase (Sigma)

3. Pasteur pipettes

4. Collagen I-coated Culture dishes

5. Scalpels, scissors, and forceps

6. Pasteur pipettes and 10-ml pipettes

7. Test tubes, 12 and 50 ml

### II. Procedures

### 2.1 Material Preparation

All materials used in this experiment must be sterile or autoclaved to prevent contamination. To enhance cell attachment to the culture dishes, collagen I-coated plates (Corning, N.Y.) must be pre-treated with the provided BSA by adding 5 ml 10 μg/ml BSA in growth media and incubated for 5 min. Aspirate the BSA solution and let the dishes air-dry in the ventilated cell culture hood for 5-10 min.

### 2.2 Surgical Specimens

Biopsies of about 1-3 mm³ are taken with biopsy forceps to sample only the mucosal layer and not the muscle layer. Surgical specimens from involved segments of the large intestine should be immediately placed in HBSS (Hank's balanced salt solution; 8.0 g/L NaCl; 0.4 g/L KCl; 0.06 g/L Na₂HPO₄×2H₂O; 0.06 g/L K₂HPO₄; 1 g/L glucose; 0.35 g/L NaHCO₃; 4.8 g/L HEPES; pH 7.2), transported on ice to the laboratory within 1 h, and worked up immediately. With autoclaved scalpels, scissors, and forceps, carefully remove muscle and fat from specimens, followed by the washing procedures. Place specimens in a 10 ml falcon tube containing 5 ml Colonic Tissue Washing Medium, followed by inoculating for 10 min at room temperature. For large tissue specimens, 50 ml falcon tubes and more wash medium is needed to ensure thorough washing. Aspirate the washing medium and repeat the washing procedures with fresh washing medium two more times. Wash tissue specimens sequentially in 70% ethanol for 1 min at the room temperature, in PBS for 5 min, and in fresh Colonic Tissue Washing Medium for 5 min. Collect tissue specimen by centrifugation prior to tissue dissociation procedures (see below).

### 2.3 Tissue Preparation and Dissociation

### 2.3.1 Human Colon OptiTDS™

In the primary cell culture, there are several important factors that can affect the yield and viability of cells, including type of tissues, origin of species, age of the animal used, enzymes, culture media and growth supplements. The Colonic Tissue Dissociation System, OptiTDS™, is suited for optimal dissociation of normal adult human biopsy samples to yield the maximum number of single primary cells of colonic tissues.

### 2.3.2 Enzyme Compositions

Collagenase I: from Clostridium Histolyticum

Collagenase III: from Clostridium Histolyticum

Collagenase IV: from Clostridium Histolyticum

Collagenase: from Clostridium Histolyticum

Trypsin: from Bovine Pancreas

### 2.3.3 System Components

Colonic Tissue Dissociation System, OptiTDS™, 2 vials.

Colon OptiTDS™ Reconstitution Buffer, (2×1 ml).

Colon OptiTDS™ Digestion Buffer, (2×9 ml).

### 2.3.4 Procedures for Tissue Preparation and Dissociation

1. Prepare fresh enzyme working solutions: to each vial of Colonic Tissue Dissociation System, OptiTDS™, add 1.0 ml of the Colon OptiTDS™ Reconstitution Buffer. Mix well.
2. Add 1.0 ml of the fresh enzyme working solution to one vial of Colon OptiTDS™ Digestion Buffer (9.0 ml). Warm the diluted enzyme working solution at 37° C. for 10 min prior to use. For optimal results, we recommend using 4-5 mg tissue samples per 10 ml diluted enzyme working solution.
3. Mince pre-washed tissue into pieces approximately 0.2-0.5 mm² in diameter with scalpels and forceps.
4. Incubate minced tissues with the diluted enzyme working solutions by incubating minced tissue samples (up to 5 mg) in 10 ml diluted enzyme working solution with slow magnetic stirring for 30 min at 37° C.
5. At the end of the tissue dissociation period, gently aspirate and expel tissue with a 10 ml pipette. This constitutes filling and emptying the barrel of the pipette at a rate of 2-3 ml per second. Repeat this procedure 5-6 times.
6. Collect cells by filtering the mixture through a cell strainer followed by centrifugation at 1×100 g. Carefully remove the medium and resuspend the cell pellet with 1.0 ml of complete culture medium.
7. Count the cells and seed in 3-4 T-25 collagen I-coated flasks (Important: pre-treat the flask with the provided BSA containing growth medium, (see below)) at the density of viable cells (2.5-5×10⁵ Cells/Dish).

### 2.3.5 Storage:

The tissue dissociation system should be reconstituted before use and can only be stored for 2-4 days at 4° C. For long-term use, it should be aliquoted and stored at −20° C. Avoid repeated freeze-thaw cycles.

### 2.4 Culture of Primary Human Colon Cells

### 2.4.1 Medium Preparation.

Thaw out the Human Colon PrimaCell™ Medium Supplements and Human Colon PrimaCell™ Serum on ice. To every 100 ml Human Colon PrimaCell™ Basal Culture Medium, add one vial of Human Colon PrimaCell™ Medium Supplements, 10 ml Human Colon PrimaCell™ Serum, and one vial of Human Colon Fibroblast Growth Inhibitors, Colon FibrOut™. Mix thoroughly and warm the complete medium in a 37° C. water bath for 10 min prior to use.

### 2.4.2 Treatment of Culture Dishes.

To enhance cell attachment to the culture dishes, collagen I-coated plates (Corning, N.Y.) must be pre-treated with the provided Coating Solution (Basal growth medium containing 10 μg/ml BSA) by adding the appropriate volume (enough to cover the whole cell-growth area) and incubating for 5 min. Aspirate the BSA solution and let the dishes air-dry in the ventilated cell culture hood for 5-10 min.

### 2.4.3 Standard Primary Culture Conditions.

Inoculate epithelial tubules and clumps of cells derived from tissue specimens into T-25 flasks coated with collagen type I, with pre-treatment of coating solution at 37° C. in a 5%-CO₂ incubator with 4 ml of complete culture medium. Change the culture medium twice weekly. The tubules and cells start to attach to the substratum, and epithelial cells migrate out within 1-2 d. Most of the tubules and small clumps of epithelium attach within 7 d, but the larger organoids can take up to 6 weeks to attach, although they will remain viable all that time.

### 2.4.4 Alternative Primary Culture Conditions.

The attachment of epithelium during primary culture and subculture is more reproducible and efficient when cells are inoculated onto collagen-coated flasks, and significantly better growth is obtained with 3T3 feeders than without. When the epithelial colonies expand to several hundred cells per colony, they become less dependent on 3T3 feeders, and no further addition of feeders is necessary. All medium and solutions described in the standard culture conditions are applicable in this culture method.

### 2.4.5 Subculture and Propagation

Most colorectal primary cultures cannot, at present, be passaged by routine trypsin/EDTA procedures. Disaggregation to single cells of the cultured colonic cells with 0.1% trypsin in 0.25 mM (0.1%) EDTA will result in extremely poor, or even no growth, so Dispase is used instead. One of the advantages of using dispase is that dispase can only detach epithelial cells but not fibroblasts, increasing the purity of epithelial cell populations.
1. Add 0.5% Dispase (Sigma, w/v) to the cell monolayer, just enough to cover the cells (˜2.5 ml/25-cm² flask), and leave the solution to stand for 40-60 min for primary cultures and 20-40 min for cell lines.
2. Once the epithelial layers begin to detach (they do so as sheets rather than single cells), pipette to help detachment and disaggregation into smaller clumps.
3. Wash and re-plate the cells under standard culture conditions. It may take several days for clumps to attach, so replace the medium carefully when feeding.

### III Fibroblast Contamination

There are several techniques published in the literature to deal with fibroblast contamination during colorectal primary cell culture. (1) Physically removing a well-isolated fibroblast colony by scraping it with a sterile blunt instrument (e.g., a cell scraper) Care has to be taken to wash the culture up to six times to remove any fibroblasts that have detached in order to prevent them from reseeding and reattaching to the flask. (2) Differential trypsinization can be attempted with the carcinomas. (3) Dispase preferentially (but not exclusively) removes the epithelium during passaging and leaves behind most of the fibroblastic cells attached to the culture vessel. During subculture, cells that have been removed with dispase can be pre-incubated in plastic petri dishes for 2-6 h to allow the preferential attachment of any fibroblasts that may have been removed together with the epithelium. This technique takes advantage of the fact that fibroblasts, in general, attach much more quickly to plastic than do clumps of melanocytes, so that a partial purification step is possible. (4) Reduce the concentration of serum to about 2.5-5% if there are heavy concentrations of fibroblastic cells. It is worth remembering that normal fibroblasts have a finite growth span in vitro and that using any or all of the preceding techniques will eventually push the cells through so many divisions that any fibroblasts will senesce.

The Human Colon PrimaCell™ system includes the Human Colon Fibroblast Growth Inhibitors, Colon FibrOut™. It contains a mixture of anti-Thy-1 monoclonal antibody, toxin ricin and formulated serum substitutes. Thy-1 antigen is present on colorectal fibroblasts but not colorectal epithelial cells; therefore, the conjugate kills contaminating fibroblasts but shows no signs of toxicity toward the epithelium whether derived from an adenoma, a carcinoma, or normal colonic tissues.

### IV References:

1. Youngman K R, Simon P L, West G A, Cominelli F, Rachmilewitz D, Klein J S, Fiocchi C, “Localisation of intestinal interleukin 1 activity and protein and gene expression to lamina propria cells”, Gastroenterology, 104:749-758 (1993).
2. Gibson P R, van de Pol E, Maxwell L E, Gabriel A, Doe W F, “Isolation of colonic crypts that maintain structural and metabolic viability in vitro”, Gastroenterology, 96:283-291 (1989).
3. Whitehead R H, Brown A, Bhathel P S, “A method for the isolation and culture of human colonic crypts in collagen gels”, In Vitro, 23:436-442 (1986).
4. Knoll N, Weise A, Claussen U, Sendt W, Marian B, Glei M, Pool-Zobel B L, “2-Dodecylcyclobutanone, a radiolytic product of palmitic acid, is genotoxic in primary human colon cells and in cells from preneoplastic lesions” Mutat Res. 594(1-2):10-19 (2005).
5. Buset M, Winawer S, Friedman E, “Defining conditions to promote the attachment of adult human colonic epithelial cells” In Vitro Cell Dev Biol. 23(6):403-412 (1987).

While this invention has been particularly shown and described with references to preferred embodiments thereof, it will be understood by those skilled in the art that various changes in form and details may be made therein without departing from the scope of the invention encompassed by the appended claims.

What is claimed is:

1. A primary cell culture system kit comprising the following components:
a) a tissue-specific composition for dissociating cells from a tissue sample;
b) a tissue-specific composition for inhibiting proliferation of contaminating cell types;
c) cell culture media;
d) supplements for cell culture media;
e) serum and/or plant additives for conditioning cell culture media; and
f) buffers for tissue preparation.

2. The kit of claim 1 further comprising instructions for using components (a) through (f).

3. The primary cell culture system kit of claim 1, wherein the tissue specific dissociation composition comprises 2 or more components selected from the group consisting of:
trypsin, collagenase, D-valine, cis-OH-proline, sodium ethylmercurithiosalicylate, phenobarbitone, fluvastatin, toxin ricin and at least one cell specific antibody.

4. The primary cell culture system kit of claim 1, wherein the tissue-specific contaminating cell inhibitory composition comprises 2 or more components selected from the group consisting of:
collagenase, collagenase I, collagenase II, collagenase III, collagenase IV, chymotrypsin, elastase, hyaluronidase, trypsin, DNase I, dispase, and papin.

5. The primary cell culture system of claim 1, wherein the media supplements comprise one or more selected from the group consisting of adenine, cholera toxin, epidermal growth factor, hydrocortisone, antibiotics, streptomycin, insulin, transferrin, highly purified bovine serum albumin, L-ascorbic acid, bovine pituitary extract, basic fibroblast growth factors, sodium selenite, 3,3′5-triiodothyronine, glutamine, dexamethasone, and cytokines.

6. The primary cell culture system of claim 1, wherein the buffers comprise one or more components selected from the group consisting of betadine solution, EDTA, EGTA, HEPES, Hank's salt solution containing fetal bovine serum, PBS, and basal culture medium containing antibiotics and serum.

7. The primary cell culture system of claim 2, wherein the instruction manual is customized for tissue samples selected from the group consisting of:
adrenal gland, kidney, bronchia, liver, bone, muscle, brain, ovary, breast, oviduct, cartilage, pancreas, cervix, pituitary gland, colon, prostate, eye, skin, fat, testis, follicles, and thymus.

8. An in vitro primary cell culture produced by the kit of claim 1.

9. A kit for promoting the enrichment and expansion of target cell types from a tissue sample, while inhibiting contaminating cell types, the kit comprising:
an enzyme composition, the enzyme composition present in amounts effective to obtain a high yield population of viable cells and
a composition that inhibits contaminating cell growth, thereby promoting the enrichment and expansion of the target cell types in culture.

10. An in vitro cell culture produced by the kit of claim 9 comprising an enriched target cell population.

11. The kit of claim 9, further comprising 1 or more components selected from the group consisting of:
culture media, culture media supplements, serum or plant additives, buffers, and instruction manuals

12. The kit of claim 9, wherein the enzyme composition comprises 2 or more components selected from the group consisting of:
trypsin, collagenase, D-valine, cis-OH-proline, sodium ethylmercurithiosalicylate, phenobarbitone, fluvastatin, toxin ricin and at least one cell specific antibody.

13. The kit of claim 9, wherein the composition comprises 2 or more components selected from the group consisting of:
collagenase, collagenase I, collagenase II, collagenase III, collagenase IV, chymotrypsin, elastase, hyaluronidase, trypsin, DNase I, dispase, and papin.

14. The kit of claim 9, wherein the contaminating cell type is fibroblast cells.

15. A method for promoting the enrichment and expansion of one or more target cell types from a tissue sample, while inhibiting contaminating cell types, the method comprising:
contacting the tissue sample with an enzyme composition, the enzyme composition present in amounts effective to obtain a high yield population of viable cells;
collecting the cell population; and
culturing the cells in the presence of a composition that inhibits contaminating cell growth, thereby enriching target cell expansion in culture.

16. An in vitro cell culture produced by the method of claim 15 comprising a substantially pure cell population with enriched target cell types.

17. The method of claim 15, wherein the contaminating cell type is fibroblast cells.

18. The method of claim 15, wherein the tissue sample is selected from a group consisting of:
adrenal gland, kidney, bronchia, liver, bone, muscle, brain, ovary, breast, oviduct, cartilage, pancreas, cervix, pituitary gland, colon, prostate, eye, skin, fat, testis, follicles, and thymus.

19. The method of claim 15, optimized for the culture of target cell types, wherein the target cell types are selected from the group consisting of:
adrenal gland cells, kidney cells, bronchial cells, liver cells, bone cells, muscle cells, brain cells, ovarian cells, mammary cells, oviduct cells, cartilage cells, pancreatic cells, cervical cells, pituitary gland cells, colon cells, prostate cells, eye cells, skin cells, fat cells, testicular cells, follicles, and thymic cells.

20. A composition for inhibiting contaminating cell growth in primary cell culture wherein the composition comprises 2 or more components selected from the group consisting of:
trypsin, collagenase, D-valine, cis-OH-proline, sodium ethylmercurithiosalicylate, phenobarbitone, fluvastatin, toxin ricin and at least one cell specific antibody.

21. The composition of claim 20, wherein the concentration of trypsin is about 0.1%-15%.

22. The composition of claim 20, wherein the concentration of collagenase is about 0.5%-12%.

23. The composition of claim 20, wherein the concentration of D-valine is about 1.0 nM-50 nM.

24. The composition of claim 20, wherein the concentration of cis-OH-proline is about 1.0 nM-3.0 mM.

25. The composition of claim 20, wherein the concentration of sodium ethylmercurithiosalicylate is about 10 nM-1.0 mM.

26. The composition of claim 20, wherein the concentration of phenobarbitone is about 1.0 nM-10 μM.

27. The composition of claim 20, wherein the concentration of fluvastatin is about 1.0 nM-1.0 μM.

28. The composition of claim 20, wherein the concentration of toxin ricin is about 1.0 nM-10 μM.

29. The composition of claim 20, further comprising a serum substitute.

30. The composition of claim 20, further comprising a buffer.

31. The composition of claim 20, wherein the cell-specific antibody comprises one or more selected from the group consisting of:
Anti-Thy-1 monclonal antibody and anti-CA125 antibody.

32. A method of inhibiting contaminating cell growth in primary cell culture, wherein the method comprises using the composition of claim 20.

33. A method of promoting target cell growth and expansion and inhibiting growth of contaminating cells, thereby enriching the target cells in a primary cell culture, comprising contacting the cell culture with 2 or more components selected from the group consisting of:
trypsin, collagenase, D-valine, cis-OH-proline, sodium ethylmercurithiosalicylate, phenobarbitone, fluvastatin, toxin ricin and at least one cell specific antibody.

34. A kit for inhibiting contaminating cell growth in primary cell culture, the kit comprising the composition of claim 33.

35. A composition for releasing one or more cell types from a tissue sample, thereby rendering the tissue sample suitable for cell culture, wherein the composition comprises 2 or more components selected from the group consisting of:
collagenase, collagenase I, collagenase II, collagenase III, collagenase IV, chymotrypsin, elastase, hyaluronidase, trypsin, DNase I, dispase, and papin.

36. The composition of claim 35, wherein the concentration of collagenase is about 0.01-10%.

37. The composition of claim 35, wherein the concentration of collagenase I is about 0.01-15%.

38. The composition of claim 35, wherein the concentration of collagenase II is about 0.01-15%.

39. The composition of claim 35, wherein the concentration of collagenase III is about 0.01-15%.

40. The composition of claim 35, wherein the concentration of collagenase IV is about 0.01-15%.

41. The composition of claim 35, wherein the concentration of chymotrypsin is about 0.03-10%.

42. The composition of claim 35, wherein the concentration of elastase is about 0.01-9%.

43. The composition of claim 35, wherein the concentration of hyaluronidase is about 0.01-15%.

44. The composition of claim 35, wherein the concentration of trypsin is about 0.5-15%.

45. The composition of claim 35, wherein the concentration of DNase I is about 0.01-5%.

46. The composition of claim 35, wherein the concentration of dispase is about 0.01-5%.

47. The composition of claim 35, wherein the concentration of papin is about 0.05-10%.

48. A method of preparing a tissue sample for cell culture, wherein the method comprises using the composition of claim 35 to release cells from the tissue sample.

49. A method of releasing cells from a tissue sample to obtain a high yield population of viable cells, the method comprising:
contacting the tissue sample with an enzyme composition comprising 2 or more components selected from the group consisting of: collagenase, collagenase I, collagenase II, collagenase III, collagenase IV, chymotrypsin, elastase, hyaluronidase, trypsin, DNase I, dispase, and papin; and
collecting the cells.

50. The method of claim 49, wherein the tissue sample is selected from a group consisting of:
adrenal gland, kidney, bronchia, liver, bone, muscle, brain, ovary, breast, oviduct, cartilage, pancreas, cervix, pituitary gland, colon, prostate, eye, skin, fat, testis, follicles, and thymus.

51. A kit for releasing cells from a tissue sample, the kit comprising the composition of claim 49.

52. The kit of claim 51, further comprising a buffer wherein the buffer comprises one or more components selected from the group consisting of:
NaCl, KCl, CaCl₂, MgCl₂, MgSO₄, Na₂HPO₄, KH₂PO₂, NaHCO₃, NaH₂PO₄, glucose, and phenol red.

53. The kit of claim 51, further comprising a buffer suitable for reconstituting lyophilized or powdered enzymes.
